(19) United States
(12) Patent Application Publication
(10) Pub. No.: US 2010/0204278 A1
(43) Pub. Date: Aug. 12, 2010

(54) Niacin Receptor Agonists, Compositions Containing Such Compounds and Methods of Treatment

(76) Inventors: Richard T. Beresis, San Francisco, CA (US); Steven L. Colletti, Princeton Junction, NJ (US)

Correspondence Address: MERCK, P O BOX 2000, RAHWAY, NJ, 07065-0907, US

(21) Appl. No.: 12/445,704

(22) PCT Filed: Oct. 16, 2007
(86) PCT No.: PCT/US07/22072

§ 371 Date: Apr. 15, 2009

Related U.S. Application Data
(60) Provisional application No. 60/853,221, filed on Oct. 20, 2006.

(51) Int. Cl. A61K 31/444; C07D 413/14; A61K 31/4439; A61P 3/10; A61P 9/10

(52) U.S. Cl. 514/333; 546/269.4; 514/338; 546/256

(57) ABSTRACT

The present invention encompasses compounds of Formula (I) as well as pharmaceutically acceptable salts and hydrates thereof, that are useful for treating atherosclerosis, dyslipidemias and the like. Pharmaceutical compositions and methods of use are also included.

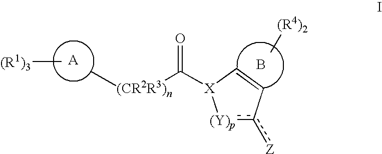

### BACKGROUND OF THE INVENTION

The present invention relates to indazole and pyrazole derivatives, compositions containing such compounds and methods of treatment or prevention in a mammal relating to dyslipidemias. Dyslipidemia is a condition wherein serum lipids are abnormal. Elevated cholesterol and low levels of high density lipoprotein (HDL) are independent risk factors for atherosclerosis associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules such as steroids and bile acids, cholesterol in excess is known to contribute to cardiovascular disease. For example, cholesterol, through its relationship with foam cells, is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

A compound represented by formula I:

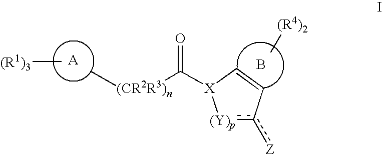

or a pharmaceutically acceptable salt or solvate thereof, wherein:

X represents a nitrogen or carbon atom;

Y represents C′ or N, such that when Y represents nitrogen, the nitrogen atom may be optionally substituted with H or R⁶ wherein:

R⁶ represents C₁₋₃ alkyl optionally substituted with 1-3 halo groups;
and when Y represents a carbon atom, the carbon atom may be substituted with hydrogen or halo;

p represents an integer of from 1 to 2, such that when p represents 2, no more than one Y represents a nitrogen atom;

the dashed lines represent optional bonds;

when the dashed line to Z represents a bond that is present, Z is selected from O, S and NH and the dashed line to (Y)ₚ represents a bond that is absent;

when the dashed line to Z represents a bond that is absent, the dashed line to (Y)ₚ represents a bond that is present and Z represents a group selected from OH, SH, NH₂, CO₂H and SO₃H;

ring B represents phenyl, a 5-7 membered carbocycle, or a 5-6 membered heteroaryl, heterocyclic or partially aromatic heterocyclic group, said heteroaryl, heterocyclic and partially aromatic heterocyclic groups containing at least one heteroatom selected from O, S and N, and optionally containing 1 additional N atom, with up to 2 heteroatoms being present;

each R⁴ is H or halo, or is selected from the group consisting of:

a) a phenyl or a 5-6 membered heteroaryl group containing 1 heteroatom selected from O, S and N, and optionally containing 1-3 additional N atoms, said phenyl and heteroaryl groups being optionally substituted with 1-3 substituents, 1-3 of which are halo, and 0-1 of which are selected from: OH, NH₂, C₁₋₃alkyl, C₁₋₃alkoxy, haloC₁₋₃ alkyl and haloC₁₋₃alkoxy; and

(b) C₁₋₃alkyl optionally substituted with 1-3 substituent groups, 1-3 of which are halo atoms, and 0-1 of which are selected from the group consisting of: OH, OC₁₋₃alkyl, NHC₁₋₃alkyl, N(C₁₋₃ alkyl)₂, CN, NO₂, Hetcy, phenyl and a 5-6 membered heteroaryl group containing 1 heteroatom selected from O, S and N, and optionally containing 1-3 additional N atoms, said phenyl and heteroaryl groups being optionally substituted with 1-3 substituents, 1-3 of which are halo, and 0-1 of which are selected from: OH, NH₂, C₁₋₃alkyl, C₁₋₃alkoxy, haloC₁₋₃alkyl and haloC₁₋₃alkoxy;

ring A represents a 6-10 membered aryl, a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present;

R² and R³ are independently H, C₁₋₃alkyl, haloC₁₋₃alkyl, OC₁₋₃alkyl, haloC₁₋₃alkoxy, OH, NH₂ or F;

n represents an integer of from 1 to 5;

each R¹ is H or is selected from the group consisting of:

a) halo, OH, CO₂H, CN, NH₂, S(O)₀₋₂R^{e} wherein R^{e} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) Hetcy, NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R″′ representing H or R″;

f) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of:
i) OH; CO₂H; CN; NH₂; S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in (b) above;
iv) NR′C(O)R″, NR′SO₂R″, NR′CO₂R″ and NR′C(O)NR″R′″ wherein

R′, R″ and R′″ are as described above.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like. With respect to the term “cycloalkenyl”, this is a subset of alkenyl.

Carbocycle is a 5-7 membered ring system containing only carbon atoms substituted with hydrogen. Ring B may be a carbocycle of from 5 to 7 atoms.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means mono-, bicyclic and tricyclic aromatic ring systems containing at least one heteroatom selected from O, S, S(O), SO₂ and N, with each ring containing 5 to 6 atoms. HAR groups may contain from 5-14, preferably 5-13 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl., quinolyl, isoquinolyl, indolyl, dihydroindolyl, quinoxalinyl, quinazolinyl, naphthyridinyl, pteridinyl, 2,3-dihydrofuro(2,3-b)pyridyl and the like. Heteroaryl also includes aromatic carbocyclic or heterocyclic groups fused to heterocycles that are non-aromatic or partially aromatic, and optionally containing a carbonyl. Examples of additional heteroaryl groups include indolinyl, dihydrobenzofuranyl, dihydrobenzothiophenyl, dihydrobenzoxazolyl, and aromatic heterocyclic groups fused to cycloalkyl rings. Examples also include the following:

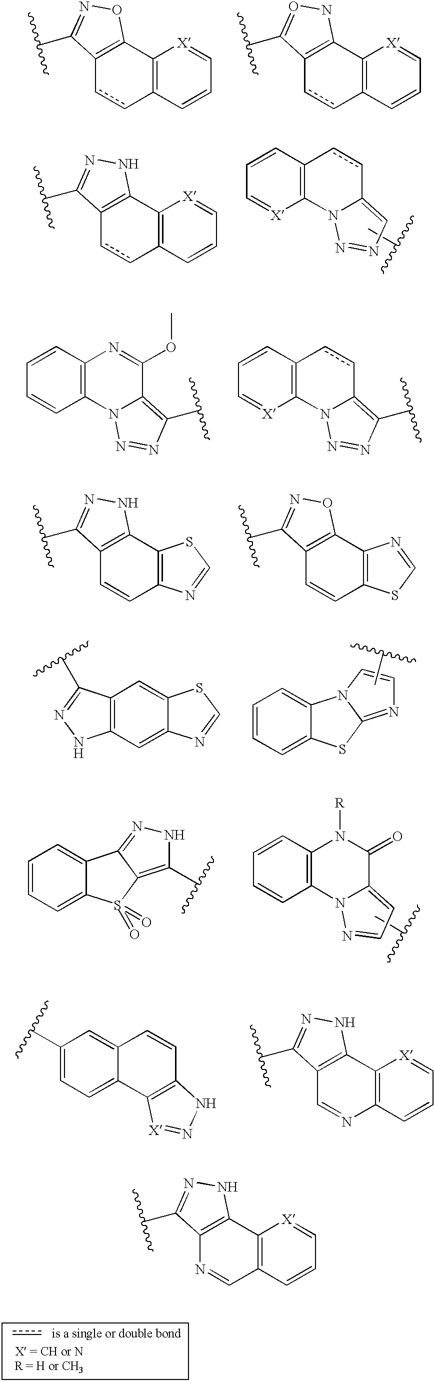

Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, tetrahydrofuranyl, 1,4-dioxanyl, morpholinyl, thiomorpholinyl, tetrahydrothienyl and the like. Heterocycles can also exist in tautomeric forms, e.g., 2- and 4-pyridones. Heterocycles moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to a compound represented by formula I:

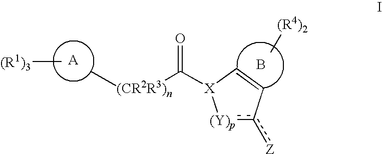

or a pharmaceutically acceptable salt or solvate thereof, wherein:

X represents a nitrogen or carbon atom;

Y represents C or N, such that when Y represents nitrogen, the nitrogen atom may be optionally substituted with H or R⁶ wherein:

R⁶ represents C₁₋₃alkyl optionally substituted with 1-3 halo groups;
and when Y represents a carbon atom, the carbon atom may be substituted with hydrogen or halo;

p represents an integer of from 1 to 2, such that when p represents 2, no more than one Y represents a nitrogen atom;

the dashed lines represent optional bonds;

when the dashed line to Z represents a bond that is present, Z is selected from O, S and NH and the dashed line to (Y)ₚ represents a bond that is absent;

when the dashed line to Z represents a bond that is absent, the dashed line to (Y)ₚ represents a bond that is present and Z represents a group selected from OH, SH, NH₂, CO₂H and SO₃H;

ring B represents phenyl, a 5-7 membered carbocycle, or a 5-6 membered heteroaryl, heterocyclic or partially aromatic heterocyclic group, said heteroaryl, heterocyclic and partially aromatic heterocyclic groups containing at least one heteroatom selected from O, S and N, and optionally containing 1 additional N atom, with up to 2 heteroatoms being present;

each R⁴ is H or halo, or is selected from the group consisting of:

a) a phenyl or a 5-6 membered heteroaryl group containing 1 heteroatom selected from O, S and N, and optionally containing 1-3 additional N atoms, said phenyl and heteroaryl groups being optionally substituted with 1-3 substituents, 1-3 of which are halo, and 0-1 of which are selected from: OH, NH₂, C₁₋₃alkyl, C₁₋₃alkoxy, haloC₁₋₃alkyl and haloC₁₋₃alkoxy; and

(b) C₁₋₃alkyl optionally substituted with 1-3 substituent groups, 1-3 of which are halo atoms, and 0-1 of which are selected from the group consisting of: OH, OC₁₋₃alkyl, NH₂, NHC₁₋₃alkyl, N(C₁₋₃alkyl)₂, CN, NO₂, Hetcy, phenyl and a 5-6 membered heteroaryl group containing 1 heteroatom selected from O, S and N, and optionally containing 1-3 additional N atoms, said phenyl and heteroaryl groups being optionally substituted with 1-3 substituents, 1-3 of which are halo, and 0-1 of which are selected from: OH, NH₂, C₁₋₃alkyl, C₁₋₃alkoxy, haloC₁₋₃alkyl and haloC₁₋₃alkoxy;

ring A represents a 6-10 membered aryl, a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present;

R² and R³ are independently H, C₁₋₃alkyl, haloC₁₋₃alkyl, OC₁₋₃alkyl, haloC₁₋₃alkoxy, OH, NH₂ or F;

n represents an integer of from 1 to 5;

each R¹ is H or is selected from the group consisting of:

a) halo, OH, CO₂H, CN, NH₂, S(O)₀₋₂R^{e} wherein R^{e} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) Hetcy, NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;

f) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of:
i) OH; CO₂H; CN; NH₂; S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in (b) above;
iv) NR′C(O)R″, NR′SO₂R″, NR′CO₂R″ and NR′C(O)NR″R′″ wherein

R′, R″ and R′″ are as described above.

A subset of compounds that is of interest relates to compounds of formula I or a pharmaceutically acceptable salt or solvate thereof wherein Y represents a nitrogen atom unsubstituted or substituted with R⁶. Within this subset, all other variables are as set forth with respect to formula I.

Alternatively, a subset of compounds that is of particular interest relates to compounds of formula I or a pharmaceutically acceptable salt or solvate thereof wherein Y represents a carbon atom. Within this subset, all other variables are as set forth with respect to formula I.

Another subset of compounds that is of interest relates to compounds of formula I or a pharmaceutically acceptable salt or solvate thereof wherein p represents 1. Within this subset, all other variables are as set forth with respect to formula I.

Another subset of compounds that is of interest relates to compounds of formula I or a pharmaceutically acceptable salt or solvate thereof wherein p represents 2. Within this subset, all other variables are as set forth with respect to formula I.

Another subset of compounds that is of interest relates to compounds of formula I or a pharmaceutically acceptable salt or solvate thereof wherein the dashed line to Z represents a bond that is present and Z represents O or the dashed line to Z represents a bond that is absent and Z represents OH. Within this subset, all other variables are as set forth with respect to formula I.

Another subset of compounds that is of interest relates to compounds of formula I or a pharmaceutically acceptable salt or solvate thereof wherein ring B represents a phenyl ring or a 5-7 membered carbocycle. Within this subset, all other variables are as set forth with respect to formula I.

More particularly, a subset of compounds that is of interest relates to compounds of formula I or a pharmaceutically acceptable salt or solvate thereof wherein ring B represents a phenyl ring. Within this subset, all other variables are as set forth with respect to formula I.

Alternatively, a subset of compounds that is of interest relates to compounds of formula I or a pharmaceutically acceptable salt or solvate thereof wherein ring B represents a 5-7 membered carbocycle. Within this subset, all other variables are as set forth with respect to formula I. Another subset of compounds that is of interest relates to compounds of formula I or a pharmaceutically acceptable salt or solvate thereof wherein ring A represents a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present. Within this subset, all other variables are as set forth with respect to formula I.

Another subset of compounds that is of interest relates to compounds of formula I or a pharmaceutically acceptable salt or solvate thereof wherein ring A represents a 5-13 membered heteroaryl group, containing at least one heteroatom selected from O, S and N, and optionally containing 1 other heteroatom selected from O and S, and optionally containing 1-3 additional N atoms, with up to 5 heteroatoms being present. Within this subset, all other variables are as set forth with respect to formula I.

More particularly, a subset of compounds that is of interest relates to compounds of formula I or a pharmaceutically acceptable salt or solvate thereof wherein ring A represents a 5 membered heteroaryl group, containing at least one heteroatom selected from O, S and N, and optionally containing 1 other heteroatom selected from O and S, and optionally containing 1-3 additional N atoms, with up to 4 heteroatoms being present. Within this subset, all other variables are as set forth with respect to formula I.

Even more particularly, a subset of compounds that is of interest relates to compounds of formula I or a pharmaceutically acceptable salt or solvate thereof wherein ring A represents a 5 membered heteroaryl group selected from the group consisting of: oxadiazole, thiazole, pyrazole, triazole and oxazole. Within this subset, all other variables are as set forth with respect to formula I.

Even more particularly, a subset of compounds that is of interest relates to compounds of formula I or a pharmaceutically acceptable salt or solvate thereof wherein ring A represents a 5 membered heteroaryl group selected from the group consisting of: oxadiazole and pyrazole. Within this subset, all other variables are as set forth with respect to formula I.

Another subset of compounds that is of interest relates to compounds of formula I or a pharmaceutically acceptable salt or solvate thereof wherein n represents 2, 3 or 4. Within this subset, all other variables are as set forth with respect to formula I.

More particularly, another subset of compounds that is of interest relates to compounds of formula I or a pharmaceutically acceptable salt or solvate thereof wherein n represents 2. Within this subset, all other variables are as set forth with respect to formula I.

Another subset of compounds that is of particular interest relates to compounds of formula I or a pharmaceutically acceptable salt or solvate thereof wherein R² and R³ are selected from the group consisting of: H, C₁₋₃alkyl, OH and NH₂, with no more than one being OH or NH₂. Within this subset, all other variables are as set forth with respect to formula I.

More particularly, another subset of compounds that is of particular interest relates to compounds of formula I or a pharmaceutically acceptable salt or solvate thereof wherein R² and R³ are selected from the group consisting of: H, C₁₋₃alkyl and NH₂, with no more than one being NH₂. Within this subset, all other variables are as set forth with respect to formula I.

Even more particularly, another subset of compounds that is of interest relates to compounds of formula I or a pharmaceutically acceptable salt or solvate thereof wherein R² and R³ are selected from the group consisting of H, CH₃ and NH₂, with no more than one being NH₂. Within this subset, all other variables are as set forth with respect to formula I.

Another subset of compounds that is of interest relates to compounds of formula I or a pharmaceutically acceptable salt or solvate thereof wherein each R¹ is H or is selected from the group consisting of:

a) halo, OH and NH₂;

b) NR′SO₂R″ wherein R′ represents H, C₁₋₃alkyl or haloC₁₋₃alkyl, and R″ represents Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups; and

c) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of OH and NH₂. Within this subset, all other variables are as set forth with respect to formula I.

In particular, another subset of compounds that is of interest relates to compounds of formula I or a pharmaceutically acceptable salt or solvate thereof wherein each R¹ is H or is selected from the group consisting of:

a) halo or OH;

b) NR′SO₂R″ wherein R′ represents H, C₁₋₃alkyl or haloC₁₋₃alkyl, and R″ represents Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups; and

c) phenyl or a 5-6 membered heteroaryl group attached at any available point and being optionally substituted with 1-3 halo, methyl or halomethyl groups, or 1 moiety selected from the group consisting of OH and NH₂. Within this subset, all other variables are as set forth with respect to formula I.

Even more particularly, another subset of compounds that is of interest relates to compounds of formula I or a pharmaceutically acceptable salt or solvate thereof wherein each R¹ is H or is selected from the group consisting of:

a) halo or OH and

b) phenyl or a 5-6 membered heteroaryl group attached at any available point and being optionally substituted with 1-3 halo, methyl or halomethyl groups, or 1 moiety selected from the group consisting of OH and NH₂. Within this subset, all other variables are as set forth with respect to formula I.

A preferred subset of compounds of the invention relates to compounds of formula I or a pharmaceutically acceptable salt thereof, wherein:

Y represents a carbon or nitrogen atom;

p represents 1 or 2, such that when p represents 2, no more than one Y represents nitrogen;

the dashed lines represent optional bonds;

when the dashed line to Z represents a bond that is present, Z represents O; and the dashed line to (Y)ₚ represents a bond that is absent, and when the dashed line to Z represents a bond that is absent, the dashed line to (Y)ₚ represents a bond that is present and Z represents OH;

ring B represents a phenyl ring or a 5-7 membered carbocycle;

ring A represents a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present;

n represents 2, 3 or 4;

each R² and R³ are selected from the group consisting of: H, C₁₋₃alkyl, OH and NH₂, with no more than one being OH or NH₂; and

each R¹ is H or is selected from the group consisting of:

a) halo, OH and NH₂;

b) NR′SO₂R″ wherein R′ represents H, C₁₋₃alkyl or haloC₁₋₃alkyl, and R″ represents Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups; and

c) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of OH and NH₂.

Examples of compounds of the present invention are set forth below in Table 1.

TABLE 1
Compound 1 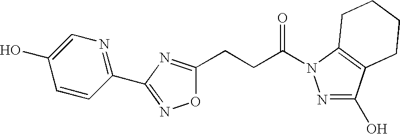
Compound 2 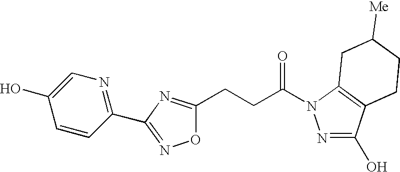
Compound 3 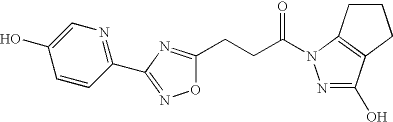
Compound 4 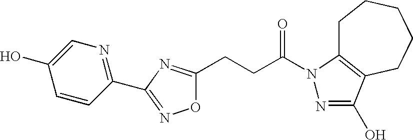
Compound 5 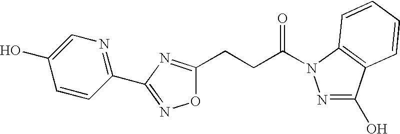
Compound 6 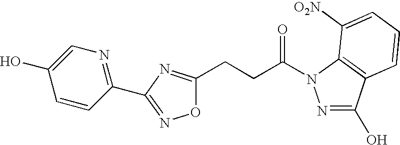
Compound 7 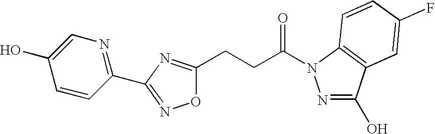
Compound 8 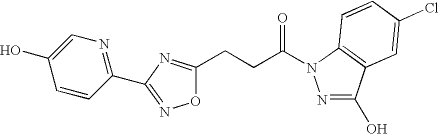
Compound 9 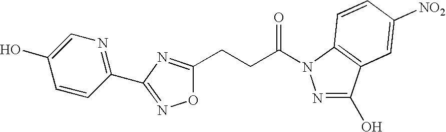
Compound 10 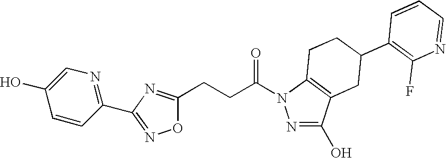
Compound 11 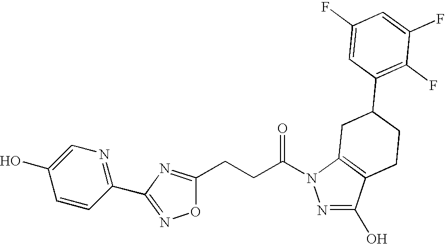
Compound 12 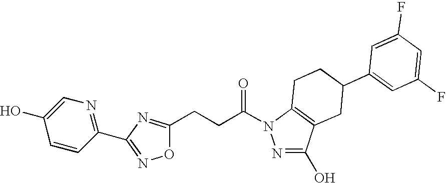
Compound 13 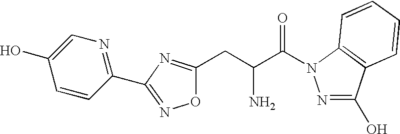
Compound 14 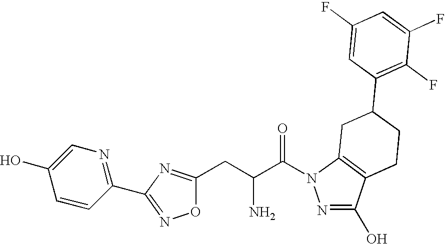
Compound 15 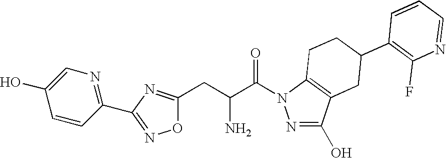
Compound 16 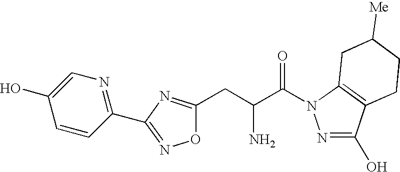
Compound 17 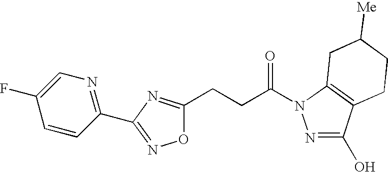
Compound 18 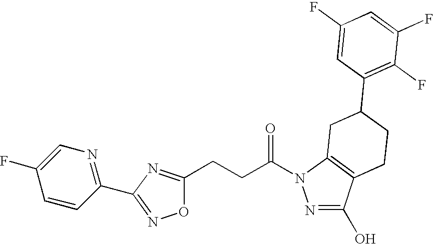
Compound 19 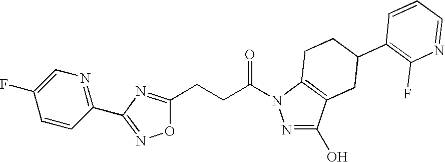
Compound 20 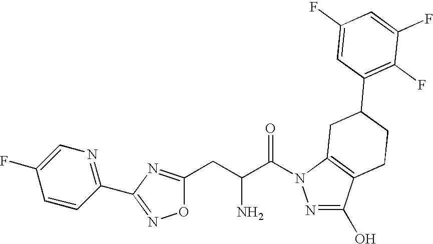
Compound 21 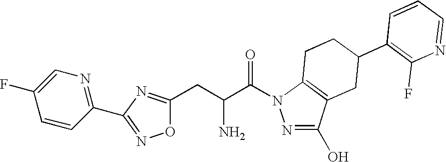
Compound 22 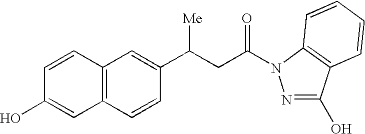
Compound 23 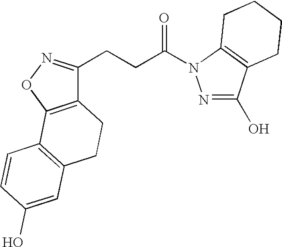
Compound 24 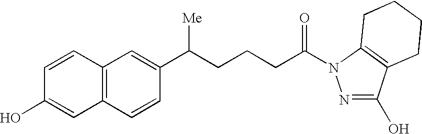
Compound 25

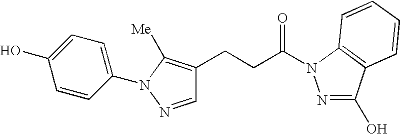

Pharmaceutically acceptable salts and solvates thereof are included as well.

Many of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula Ito an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. Tautomeric forms are also exemplified in Formula I by the dashed lines representing optional bonds. The individual tautomers as well as mixtures thereof are included.

### Dosing Information

The dosages of compounds of formula I or a pharmaceutically acceptable salt or solvate thereof vary within Wide limits. The specific dosage regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage range is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient. Examples of suitable dosage amounts include approximately 0.1 mg, 0.5 mg, 1 mg, 2 mg, 5 mg, 8 mg, 10 mg, 15 mg, 20 mg, 25 mg, 50 mg, 60 mg, 75 mg, 80 mg, 100 mg, 150 mg, 200 mg, 250 mg, 300 mg, 400 mg, 500 mg, 600 mg, 700 mg, 750 mg, 1000 mg and the like.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPAR-gamma) agonists including the compounds commonly referred to as glitazones for example pioglitazone and rosiglitazone and, including those compounds included within the structural class known as thiazolidine diones as well as those PPAR-gamma agonists outside the thiazolidine dione structural class; PPAR-alpha agonists such as clofibrate, fenofibrate including micronized fenofibrate, and gemfibrozil; PPAR dual alpha/gamma agonists; vitamin B6 (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA 1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “Dselective”. This is in accordance with US Published Application No. 2004/0229844A1 published on Nov. 18, 2004, incorporated herein by reference.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include those compounds disclosed in PCT WO2004/103370A1 published on Dec. 2, 2004.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate.

The heterocyclic acid compounds of the invention also include esters of formula I that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

### Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ disclosed in WO2004/103370A1 published on Dec. 2, 2004 in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, disclosed in WO2004/103370A1 published on Dec. 2, 2004 in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, disclosed in WO2004/103370A1 published on Dec. 2, 2004 and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventin or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

### Methods of Synthesis for Compounds of Formula I

Compounds of Formula I have been prepared by the following representative reaction schemes. It is understood that similar reagents, conditions or other synthetic approaches to these structure classes are conceivable to one skilled in the art of organic synthesis. Therefore these reaction schemes should not be construed as limiting the scope of the invention. All substituents are as defined above unless indicated otherwise.

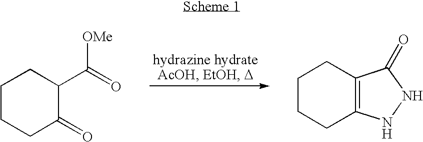

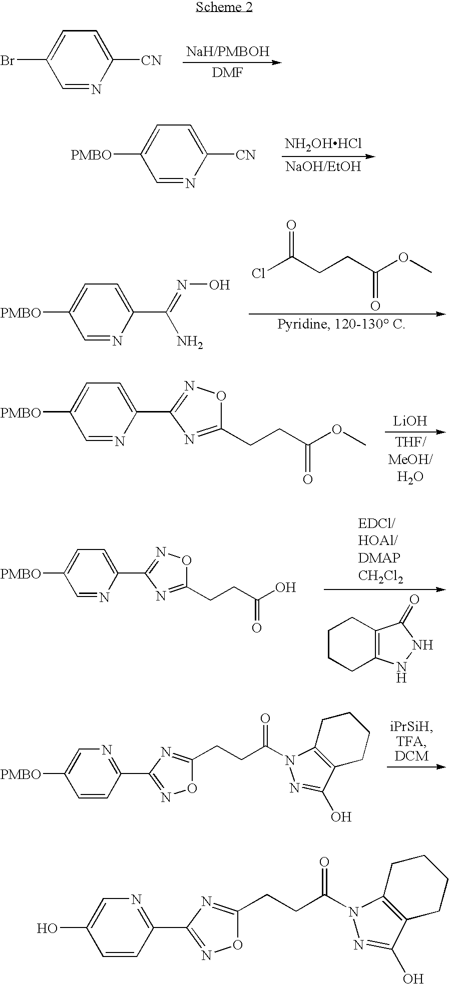

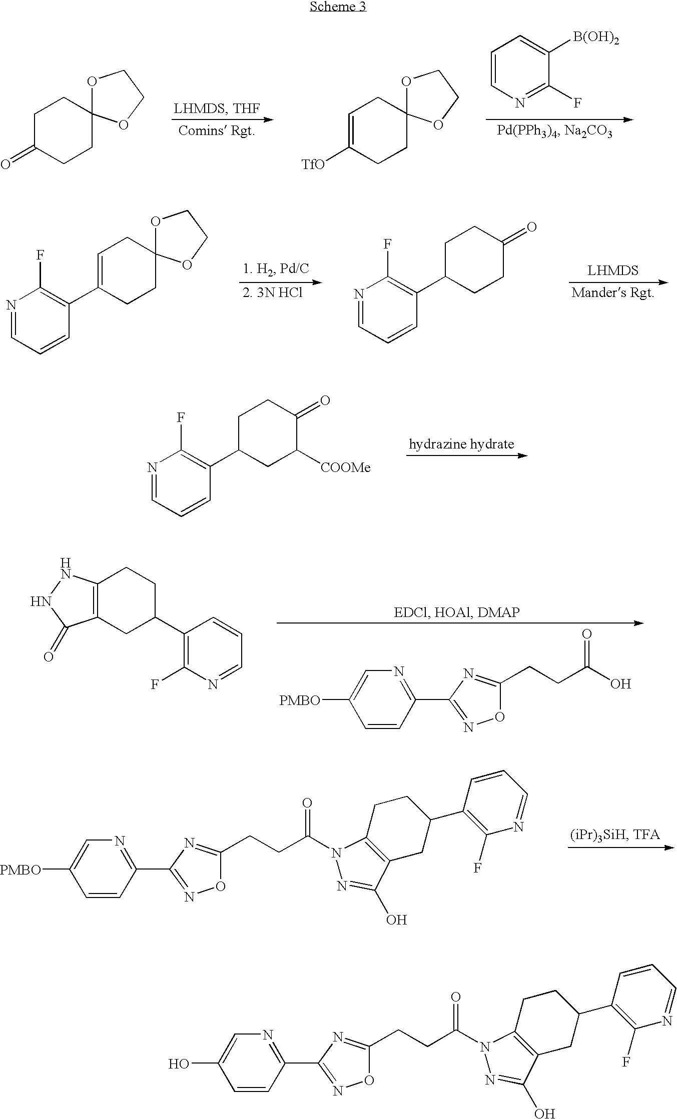

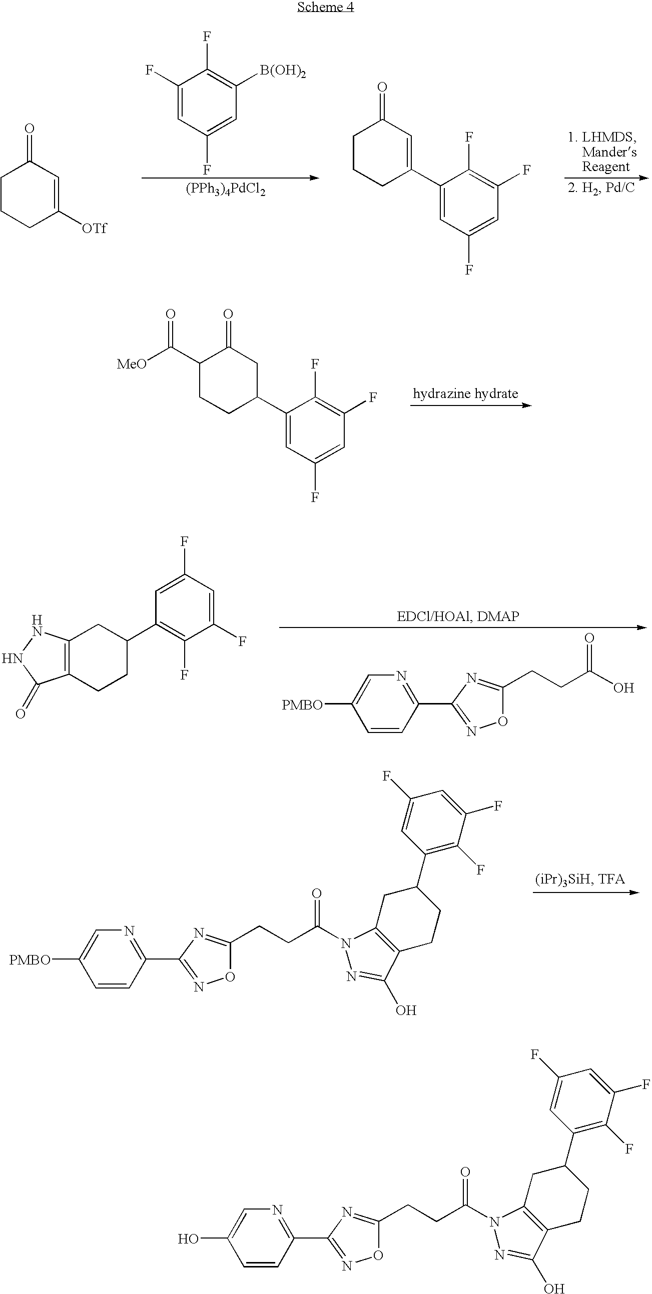

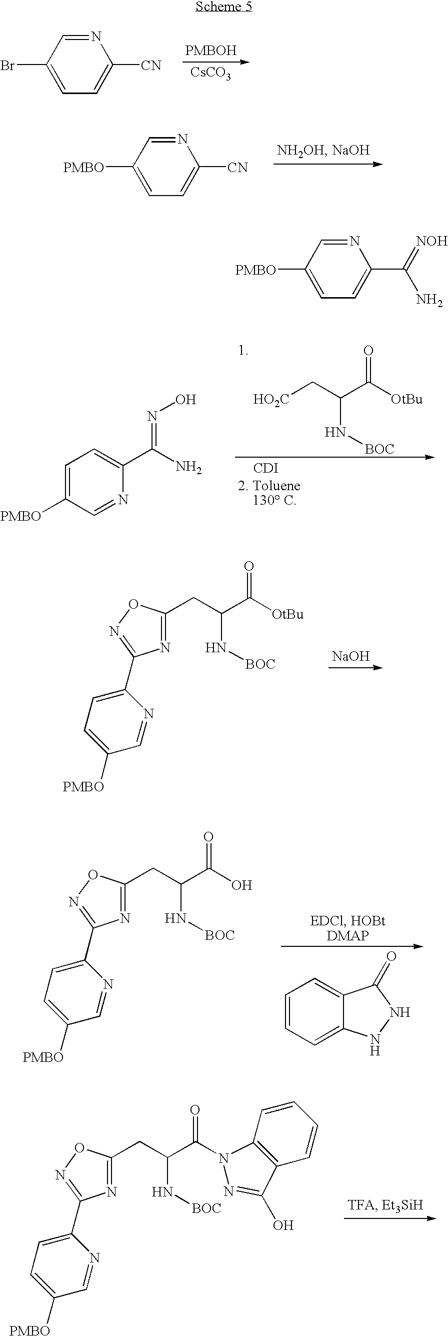

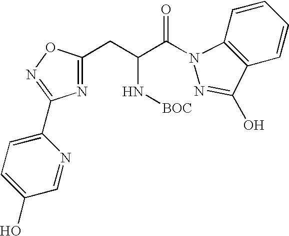

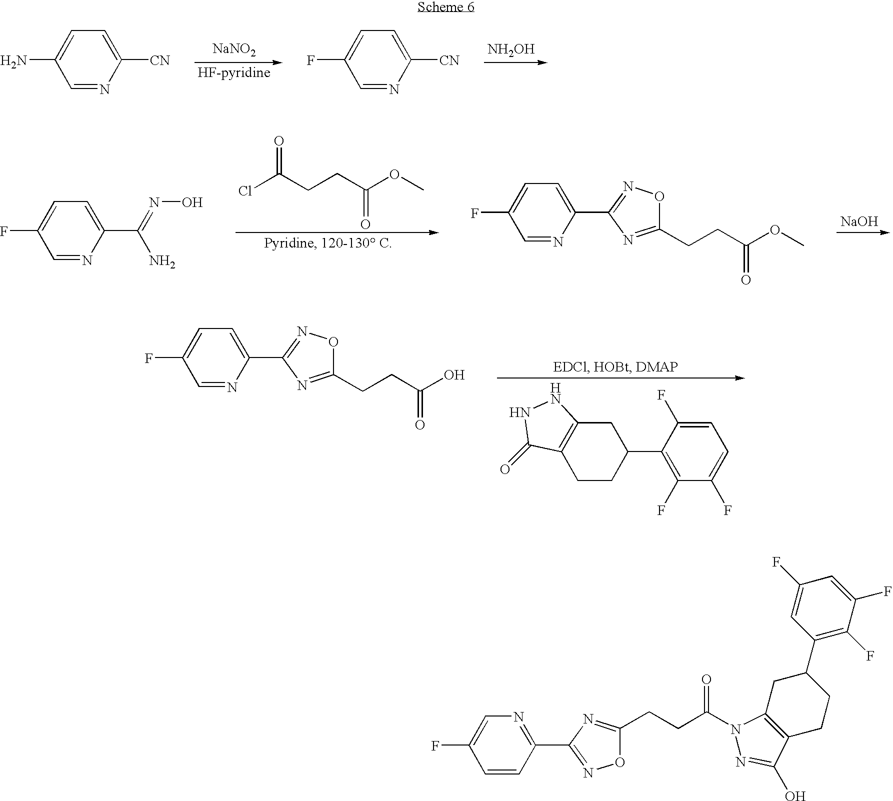

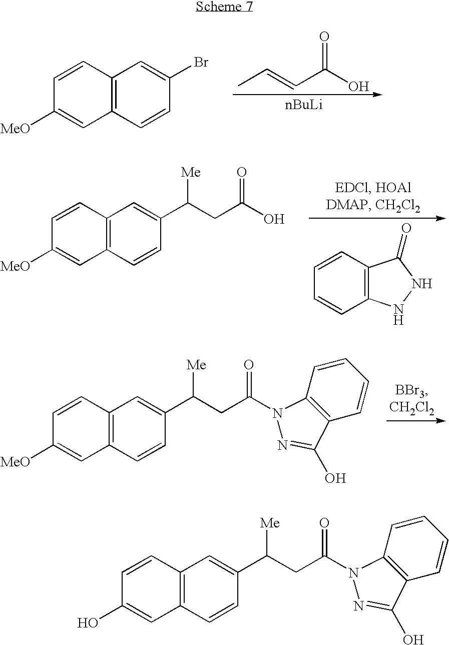

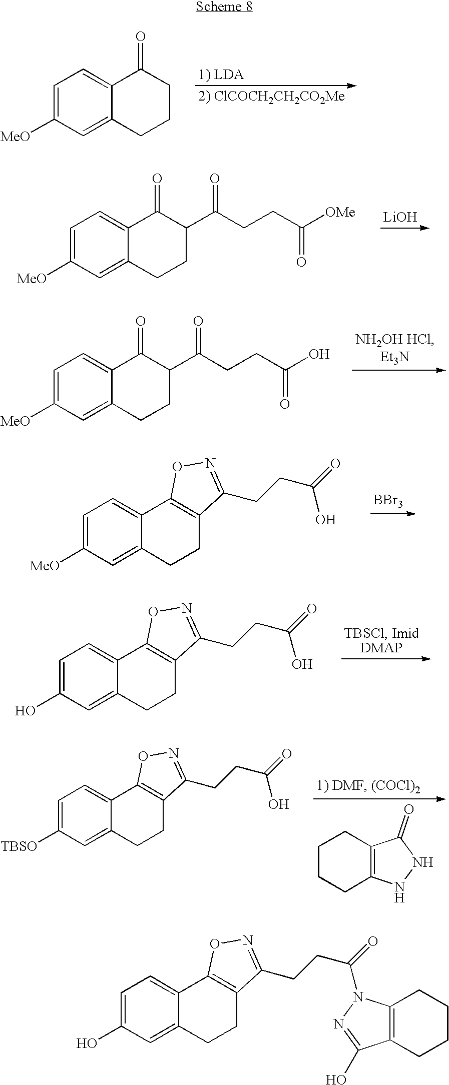

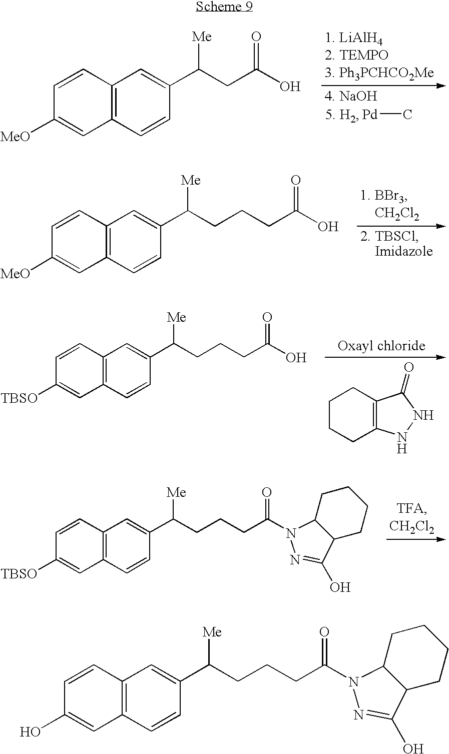

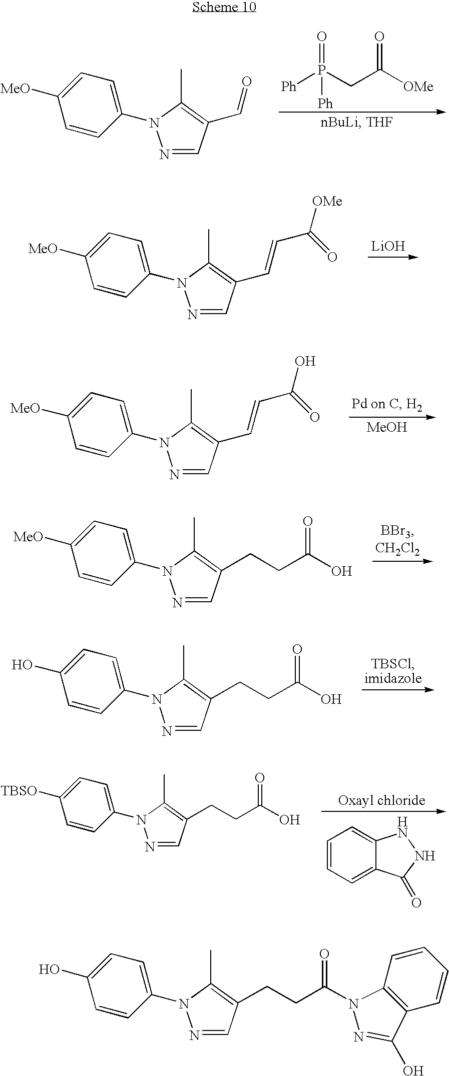

### REPRESENTATIVE EXAMPLES

The following examples are provided to more fully illustrate the present invention, and shall not be construed as limiting the scope in any manner. Unless stated otherwise:

(i) all operations were carried out at room or ambient temperature (rt or RT), that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (¹H NMR) spectrometry, and the purity was assured by at least one of the following techniques: TLC or HPLC;

(vi) ¹H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid−acetonitrile, A=0.1% formic acid−water), Waters XTerra C18˜3.5 um-50×3.0 mmID and diode array detection;

(viii) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(ix) the manual purification of compounds by preparative reverse phase HPLC (RPHPLC) was conducted on either a Waters Symmetry Prep C18-5 um-30×100 mmID, or a Waters Atlantis Prep dC18-5 um-20×100 mmID; 20 mL/min, 10-100% B linear gradient over 15 min (B=0.05% TFA-acetonitrile, A=0.05% TFA-water), and diode array detection;

(x) the purification of compounds by preparative thin layer chromatography (PTLC) was conducted on 20×20 cm glass prep plates coated with silica gel, commercially available from Analtech;

(xi) flash column chromatography was carried out on a glass silica gel column using Kieselgel 60, 0.063-0.200 mm (SiO₂), or a Biotage SiO₂ cartridge system including the Biotage Horizon and Biotage SP-1 systems;

(xii) chemical symbols have their usual meanings, and the following abbreviations have also been used: h (hours), min (minutes), v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar);

(xiii) definitions of acronyms and abbreviations are as follows:

| | |
|---|---|
| Comins' Reagent is 2-[N,N- | CDI is 1,1′-carbonyl diimidazole |
| Bis(trifluromethylsulfonyl)amino]- | PMB is para-methoxybenzyl |
| 5-chloropyridine | DMAP is 4-dimethyl amino pyridine |
| DCM is dichloromethane | EDCI is 1-ethyl-3-(3-dimethylamino- |
| (methylene chloride) | propyl)-carbodiimide hydrochloride |
| DMF is dimethylformamide | LDA is lithium diisopropyl amide |
| DMSO is dimethyl sulfoxide | Mander's Reagent is methyl |
| THF is tetrahydrofuran | cyanoformate |
| LHMDS is lithium bis(trimethyl- | Pd(PPh₃)₄ is tetrakis triphenyl- |
| silyl) amide | phosphine palladium (0) |
| OTf is triflate | HOAt is 1-hydroxy-7- |
| TEMPO is 2,2,6,6-tetramethyl-1- | azabenzotriazole |
| piperidinyloxy, free radical | TBSOTf is t-butyl dimethyl silyl |
| TBSC is t-bulyl dimethyl silyl | trifluoromethane sulfonate |
| chloride | |
| TFA is trifluoroacetic acid | |

### INTERMEDIATE A

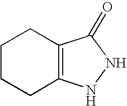

Methyl 2-oxocyclohexane carboxylate (1.56 g, 10 mmol) was dissolved in 5 mL of dry ethanol and hydrazine hydrate (15 mmol, 0.47 mL) was added. The resulting solution was heated to reflux for 15 hours. Upon cooling to rt, the desired product precipitates as a white solid, which was filtered and washed with cold ethanol giving the pure product intermediate A as defined in Scheme 1.

### Example 1

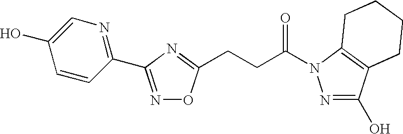

As shown in Scheme 2, NaH (7.2 g, 60%) was added to DMF (100 mL) followed by 4-methoxybenzyl alcohol (18.7 mL) at 0° C. After 25 min at 0° C., the mixture was warmed to 23° C. and stirred for an additional 30 min. To the resulting solution was added the pyridyl cyanobromide (22.9 g) in one portion. The reaction was exothermic and stirred for 10 min before it was cooled to room temperature. The mixture was diluted with 500 mL of ethyl acetate, washed with water (500 mL×3). The first two aqueous phases were extracted with dichloromethane (500 mL×2). The combined dichloromethane phase was washed with water (500 mL×3). The combined organic phases were dried over sodium sulfate and concentrated to give the PMB ether as a white solid.

To the suspension of this intermediate (24.6 g) and hydroxylamine hydrochloride (8.55 g) in ethanol (500 mL) was added NaOH (4.92 g in 50 mL of water) dropwise. The mixture was stirred at RT overnight. The solid was collected by filtration to give the N-hydroxy amidine as a white solid.

To this amidine intermediate (15.4 g) was added pyridine (40 mL) and the acid chloride shown in Scheme 2 (8.3 mL). The mixture was heated at 120° C. for 2 h and then 130° C. for 1 h. After removing most pyridine, the residue was partitioned between water and dichloromethane. The organic phase was washed with water four times and then dried with sodium sulfate. After removing the solvent, to the residue was added some methanol. The resulting slurry was filtered. The solid collected by the filtration was washed with methanol and dried in vacuo to give the methyl ester intermediate as a pale pink solid.

To this ester (30 g) suspended in 3:1:1 THF/MeOH/water (700 mL) was added LiOH (300 mL, 1 N). The mixture was stirred at RT for 1 h. After removing most of the solvent, the aqueous layer was acidified to pH=3. Filtration of the resulting slurry gave a white solid, which was washed with water, diethyl ether and azeotroped with toluene to give the acid as a white solid.

Intermediate A (43 mg, 0.28 mmol) and the carboxylic acid (100 mg, 0.28 mmol) intermediate described above were dissolved in dichloromethane and cooled to 0° C. HOAt (57 mg, 0.42 mmol), EDCI (81 mg, 0.42 mmol) and DMAP (5 mg) were added and the resulting reaction mixture was gradually warmed to rt over 15 hours. The reaction mixture was then diluted with water and extracted with ethyl acetate. The organic layers were combined and evaporated and the resulting residue was purified by reverse phase HPLC, giving the desired ester intermediate (106 mg) as a white solid.

This ether intermediate (12 mg, 0.026 mmol) was dissolved in DCM cooled to 0° C., and (0.5 mL), TFA (0.25 mL) and triisopropyl silane (0.125 mL) were added. The reaction mixture was held at 0° C. for 1 hour, and then evaporated under reduced pressure. The residue was purified by reverse phase HPLC giving EXAMPLE 1 as defined in Scheme 2. NMR (DMSO-d₆, 500 MHz) δ 10.95 (1H, s), 10.61 (1H, s), 8.25 (1H, s), 7.87 (1H, d), 7.29 (1H, dd), 3.51 (2H, t), 3.27 (t, 3H), 2.79 (br s, 2H), 2.23 (br s, 2H), 1.67-1.61 (m, 4h); LCMS m/z 356 (M+1).

### Example 2

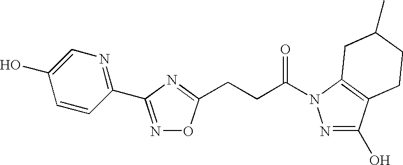

EXAMPLE 2 was prepared in a similar manner to EXAMPLE 1 (Scheme 2) utilizing the intermediate prepared from commercially available ethyl-4-methyl-2-cyclohexanone (Scheme 1). NMR (DMSO-d₆, 500 MHz) δ 10.96 (1H, s), 10.61 (1H, s), 8.25 (1H, s), 7.87 (1H, d), 7.30 (1H, d), 3.48 (2H, m), 3.26 (211, t), 3.02 (1H, dd), 2.30 (2H, br m), 2.22 (1H, br m), 1.73 (2H, br m), 1.23 (1H, br m), 0.99 (3H, d); LCMS m/z 370 (M+1).

### Example 3

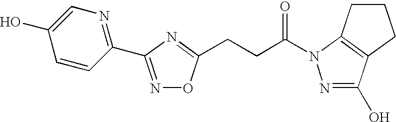

EXAMPLE 3 was prepared in a similar manner to EXAMPLE 1 (Scheme 2) utilizing the intermediate prepared from commercially available methyl 2-oxocyclopentane carboxylate (Scheme 1). NMR (CD₃OD, 500 MHz) δ 8.25 (d, 1H), 7.87 (d, 1H), 7.29 (dd, 1H), 3.46 (t, 2H), 3.29 (t, 2H), 2.83 (br s, 2H), 2.49-2.48 (m, 2H), 2.48-2.41 (m, 2H); LCMS m/z 342 (M+1).

### Example 4

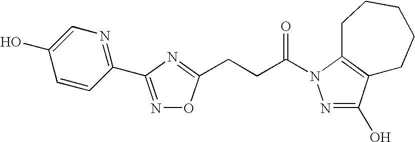

EXAMPLE 4 was prepared in a similar manner to EXAMPLE 1 (Scheme 2) utilizing the intermediate prepared from commercially available methyl 2-oxocycloheptane carboxylate (Scheme 1). ¹H NMR (DMSO-d₆, 500 MHz) δ 10.92 (br s, 1H), 10.59 (br s, 1H), 8.25 (d, 1H), 7.87 (d, 1H), 7.29 (dd, 1H), 3.54 (t, 2H), 3.25 (t, 2H), 3.17 (t, 2H), 2.35 (t, 2H), 1.73-1.71 (m, 2H), 1.62 (br s, 2H), 1.58-1.57 (m, 2H); LCMS m/z 370 (M+1).

### Example 5

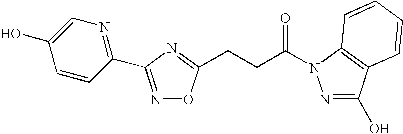

EXAMPLE 5 was prepared in a similar manner to EXAMPLE 1 (Scheme 2) utilizing the commercially available 3-indazolinone. ¹H NMR (CD₃OD, 600 MHz) δ 8.24 (d, 1H), 8.19 (s, 1H), 7.94 (d, 1H), 7.70 (d, 1H), 7.54 (t, 1H), 7.33 (t, 1H), 7.29 (dd, 1H), 3.69 (t, 2H), 3.41 (t, 2H); LCMS m/z 352 (M+1).

### Example 6

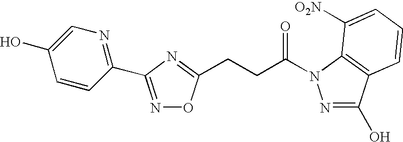

EXAMPLE 6 was prepared in a similar manner to EXAMPLE 1 (Scheme 2) from commercially available 7-nitro-1,2-dihydro-3H-indazol-3-one. ¹H NMR (acetone-d₆, 600 MHz) δ 8.58 (d, 1H), 8.33-8.31 (m, 2H), 7.94 (d, 1H), 7.46 (t, 1H), 7.38 (d, 1H), 3.81 (t, 2H), 3.49 (t, 2H), LCMS m/z 397 (M+1).

### Example 7

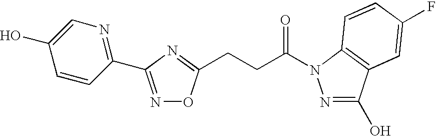

Sodium nitrite (1.2 g, 16.5 mmol) was dissolved in 2.5 mL of water and the resulting solution was added dropwise to 2-amino-5-fluorobenzoic acid (2.6 g, 16.5 mmol) in 3 mL of concentrate aqueous HCl and 15 mL of water at 0° C. The reaction mixture was allowed to stir for 30 minutes before sodium sulfite (5.7 g, 450 mmol) in 15 mL of water was added in one portion. This solution was then stirred for 2 hours before adding 5 mL of concentrated aqueous HCl. The reaction mixture was continuously stirred at rt for 48 hours and the precipitated white solid was filtered and washed with methanol providing the desired 6-fluoro-1,2-dihydro-3H-indazol-3-one.

EXAMPLE 7 was prepared in a similar manner to EXAMPLE 1 (Scheme 2) from 6-fluoro-1,2-dihydro-3H-indazol-3-one synthesized as described above. ¹H NMR (DMSO-d₆, 500 MHz) δ 8.21 (d, 1H), 7.97 (d, 1H), 7.52-7.46 (m, 2H), 7.33-7.27 (m, 2H), 7.74 (t, 2H), 3.44 (t, 2H); LCMS m/z 370 (M+1).

### Example 8

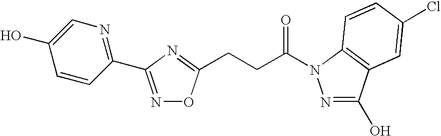

EXAMPLE 8 was prepared in a similar manner to EXAMPLE 7 (Scheme 2) from commercially available 2-amino-5-chlorobenzoic acid. NMR (DMSO-d₆, 500 MHz) δ12.39 (s, 1H), 10.60 (s, 1H), 8.24 (d, 1H), 8.21 (d, 1H), 7.87 (d, 1H), 7.83 (d, 1H), 7.63 (dd, 1H), 7.28 (dd, 1H), 3.64 (t, 1H), 3.38 (t, 2H); LCMS m/z 386 (M+1).

### Example 9

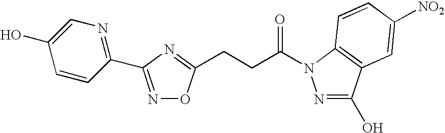

EXAMPLE 9 was prepared in a similar manner to EXAMPLE 7 (Scheme 2) from commercially available 2-amino-5-nitrobenzoic acid. ¹H NMR (DMSO-d₆, 500 MHz) δ 9.11 (d, 1H), 8.23-8.20 (m, 2H), 7.97 (d, 1H), 7.93 (d, 1H), 7.31 (dd, 1H), 3.75 (t, 2H), 3.46 (t, 2H; LCMS m/z 397 (M+1).

### Example 10

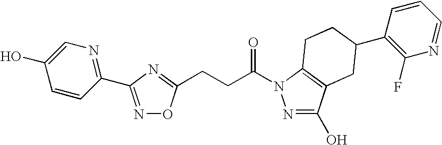

As shown in Scheme 3 a solution of 1,4-cyclohexane dione mono-ethylene ketal (4.0 g, 25.6 mmol) in anhydrous THF (130 mL) cooled to −78° C. under a N₂ atmosphere was added LHMDS (28 mL, 28 mmol, 1.0 M in THF). After stirring for 1 hour a solution 2-[N,N-Bis (trifluoromethylsulfonyl)amino]-5-chloropyridine (10.0 g, 25.4 mmol) in THF (100 mL) was added. The reaction mixture was warmed to room temperature and stirred for 18 hours, quenched with water, and the resulting mixture was extracted with ethyl acetate (3×). The combined organic layers were washed with brine, dried over anhydrous sodium sulfate, filtered and concentrated in vacuo. The residue was purified by flash chromatography (Biotage, Horizon) using (0% EtOAc/Hexane→20% EtOAc/Hexane) to give the desired triflate product as a colorless oil.

To a solution of the triflate intermediate (7.00 g, 24.2 mmol) in THF (200 mL) was added 2-fluoro-3-pyridine boronic acid (3.42 g, 24.2 mmol), and tetrakis triphenyl phosphine palladium (0) (1.00 g, 0.9 mmol). Aqueous sodium carbonate solution (1M, 48 mL) was added, the reaction mixture was flushed with N₂ and heated to 50° C. for 1 hour. The mixture was cooled to room temperature, diluted with ethyl acetate, washed with brine, and dried over sodium sulfate. The crude material was purified by flash chromatography (Biotage Horizon) (20% EtOAc/Hexane→40% EtOAc/Hexane) to give the desired fluoro pyridine product.

To a solution of the fluoro pyridine intermediate (5.71 g, 24.3 mmol) in MeOH (10 mL) was added palladium on carbon (5%, 2 g) in MeOH (10 mL). The reaction mixture was stirred under a hydrogen balloon for 18 hours, and then filtered through celite and concentrated in vacuo. The crude material was dissolved in THF/EtOH (100 mL/40 mL) and HCl (80 mL, 3N) was added. The resulting mixture was stirred at room temperature for 18 hours. The reaction mixture was concentrated in vacuo. The residue was diluted with ethyl acetate, and adjusted to pH=8 with 1 N NaOH. The resulting mixture was extracted with EtOAc (2×), washed with brine and dried over Na₂SO₄, filtered and concentrated in vacuo. The crude material was purified by flash chromatography (Biotage Horizon) (0% EtOAc/Hexane 60% EtOAc/Hexane) to give the desired ketone product.

To a solution of the ketone intermediate (1.18 g, 6.11 mmol) in anhydrous THF (61 mL) cooled to −78° C. under a N₂ atmosphere was added LHMDS (6.16 mL, 9.16 mmol, 1.0 M in THF). After 1 hour, Mander's Reagent (0.686 mL, 8.54 mmol) was added, and the mixture was warmed to −40° C. over 2 hours. The reaction mixture was quenched with 1N HCl and extracted with EtOAc (2×). The organic layer was washed with brine and dried over Na₂SO₄, filtered and concentrated in vacuo. This keto ester product without any further purification was converted to the intermediate as described in Scheme 1 for Intermediate A.

EXAMPLE 10 was prepared in a similar manner to EXAMPLE 1 (Scheme 2) from the above intermediate. ¹H NMR (DMSO-d₆, 500 MHz) δ 8.25 (d, 1H), 8.10 (d, 1H), 7.92-7.87 (m, 2H), 7.33-7.29 (m, 2H), 3.54-2.47 (m, 2H), 3.31-3.28 (m, 2H), 3.07 d, 1H), 2.89-2.83 (m, 1H), 2.58 (dd, 1H), 1.99-1.87 (m, 2H); LCMS m/z 451 (M+1).

### Example 11

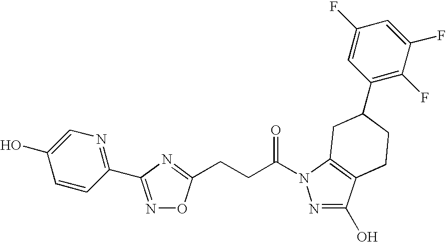

A solution of cyclohexane 1,3-dione (1.0 g, 8.92 mmol) and 2,6-lutidine (2.07 mL, 17.84 mmol) in DCM cooled to 0° C. was treated with trifluoromethane sulfonic anhydride (2.25 mL, 13.38 mmol). The reaction mixture was stirred at room temperature for 30 minutes and quenched by the addition of 1N HCl. The resulting mixture was extracted with DCM. The organic layer was washed with 1N HCl, dried over anhydrous sodium sulfate, filtered and concentrated in vacuo. The residue was purified by flash chromatography using 20% ethyl acetate hexanes to give the desired triflate product as a light brown oil.

To a solution of this triflate (8.71 g, 35.7 mmol) in THF (100 mL) was added 2,3,5-trifluorophenyl boronic acid, Na₂CO₃ (50 mL, 2.0 M solution) and dichlorobis (triphenylphosphine)-palladium (1.0 g). The resulting mixture was heated at 60° C. under a nitrogen atmosphere. After 30 minutes, the reaction mixture was cooled to room temperature and diluted with ethyl acetate. The organic layer was washed with brine, dried over anhydrous sodium sulfate, filtered and concentrated in vacuo. The residue was purified by flash chromatography using 10% ethyl acetate hexanes to give the desired trifluorophenyl compound as a light yellow solid.

To a solution of this trifluorophenyl derivative (7.5 g, 33.2 mmol) in anhydrous THF cooled to −78° C. under a nitrogen atmosphere was added LHMDS (36.5 mL, 36.5 mmol, 1.0 M in THF). The reaction mixture was stirred at 0° C. for 25 minutes. It was then cooled to −78° C. and methyl cyano formate (3.16 mL, 39.78 mmol) was added. After 30 minutes, the reaction was quenched by pouring into water (100 mL). The resulting mixture was extracted with ethyl acetate (3×). The organic layer was washed with brine dried over anhydrous sodium sulfate, filtered and concentrated in vacuo. The residue was purified by flash chromatography (silica-gel) using 10% ethyl acetate-hexanes to give the desired keto ester product as a yellow solid.

To a solution of this keto ester intermediate (7.49 g, 26.4 mmol) in methanol (100 mL) was added Pd/C (100 mg, 10% by weight). The resulting reaction was stirred under H₂ balloon for 18 hours. The reaction mixture was filtered through celite. The filtrate was concentrated in vacuo and purified by flash chromatography using 10% ethyl acetate-hexanes to give the desired saturated product as a colorless oil (Scheme 4). This keto ester product without any further purification was converted to the intermediate as described in Scheme 1.

EXAMPLE 11 was prepared in a similar manner to EXAMPLE 1 (Scheme 2) from the above intermediate. ¹H NMR (DMSO-d₆, 500 MHz) δ 8.25 (s, 1H), 7.87 (d, 1H), 7.40-7.37 (m, 1H), 7.31 (dd, 1H), 7.15-7.13 (m, 1H), 3.56-3.48 (m, 2H), 3.27-3.19 (m, 4H), 2.84 (q, 1H); LCMS m/z 486 (M+1).

### Example 12

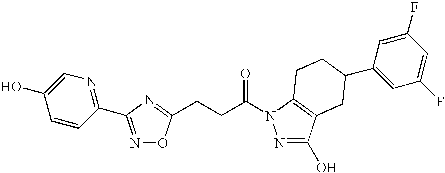

EXAMPLE 12 was prepared in a similar manner to EXAMPLE 11 starting from commercially available 3,5-difluorophenyl boronic acid (Scheme 4). ¹H NMR (DMSO-d₆, 500 MHz) δ 11.06 (br s, 1H), 10.59 (br s, 1H), 8.25 (d, 1H), 7.88 (d, 1H), 7.30 (dd, 1H), 7.06 (d, 2H), 3.52 (t, 2H), 3.29 (t, 2H), 3.06 (d, 1H), 2.91 (br s, 1H), 2.82 (br s, 1H), 2.56 (d, 1H), 2.37 (d, 1H), 1.97 (br s, 1H), 1.88-1.85 (m, 1H); LCMS m/z 468 (M+1).

### Example 13

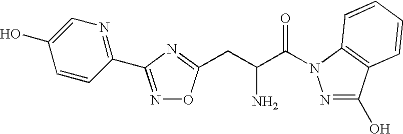

As shown in Scheme 5 a mixture of 5-bromo-2-cyanopyridine (1 g, 5.5 mmol), cesium carbonate (3.6 g, 11 mmol), 4-methoxybenzyl alcohol (1.5 g, 10.9 mmol) in a solution of 20 mL of toluene, was quickly added 1,10-phenanthroline (98 mg, 0.55 mmol) and copper(I) iodide (52 mg, 0.27 mmol) under nitrogen. The reaction mixture was heated at 120° C. overnight. To the mixture was then added water (150 mL), and partitioned twice with ethyl acetate (2×100 mL). The aqueous layer was then extracted twice with dichloromethane (2×100 mL). The combined organic phases were dried with sodium sulfate and concentrated in vacuo. The residue was dissolved in DMSO and purified by RPHPLC to give 5-(4-methoxybenzyloxy)-2-cyanopyridine as a pale yellow solid. To a slurry of this intermediate (60 mg, 0.25 mmol) and hydroxylamine hydrochloride (38 mg, 0.55 mmol) in 8 mL of ethanol, was added 0.17 mL of 3 N sodium hydroxide aqueous solution. The reaction mixture was stirred at 23° C. overnight. The residue was purified by RPHPLC to give 5-(4-methoxybenzyloxy)-2-hydroxyamidinylpyridine as a white solid. To the commercially available Boc-tert-butoxy-aspartic acid (10.0 g, 35 mmol) in CH₂Cl₂ (100 mL) was added CDI (11 g, 69 mmol). The reaction mixture was stirred at room temperature for 1 hour and then the corresponding N′-hydroxy-pyridinecarboximidamide prepared above (19.0 g, 69 mmol) was added. The reaction mixture was allowed to stir for 2 hours, at which time it was filtered, and the organic layer was washed with saturated ammonium chloride (100 mL), dried over sodium sulfate, and concentrated in vacuo. Without further purification, the aspartic acid derivative (5.0 g, 9.1 mmol) in toluene (50 mL) was heated at 130° C. for 16 hours. The mixture was concentrated in vacuo and purified via flash chromatography (Biotage 40M). To a solution of the oxadiazole (3.71 mg, 7.0 mmol) in 50 mL of THF/MeOH/H₂O (2:5:1), was added sodium hydroxide (0.84 g, 21 mmol). The biphasic solution was allowed to stir for 12 h. The mixture was concentrated in vacuo, diluted with 10 mL of water, cooled to 0° C. and acidified with concentrated HCl to a pH of 3. The acidic solution was extracted three times with ethyl acetate (20 mL) and the organic extracts were dried with sodium sulfate and concentrated in vacuo, giving the desired carboxylic acid.

EXAMPLE 13 was prepared (Scheme 5) from the above carboxylic acid derivative and commercially available 3-indazolinone in a manner similar to EXAMPLE 1 (Scheme 2). ¹H NMR (DMSO-d₆, 500 MHz) δ 8.84 (br s, 2H), 8.28 (d, 1H), 8.26 (d, 1H), 7.83 (d, 1H), 7.75 (d, 1H), 7.71 (t, 1H), 7.47 (t, 1H), 7.28 (d, 1H), 5.41 (q, 1H), 3.85 (dd, 1H), 3.74 (dd, 1H); LCMS m/z 367 (M+1).

### Example 14

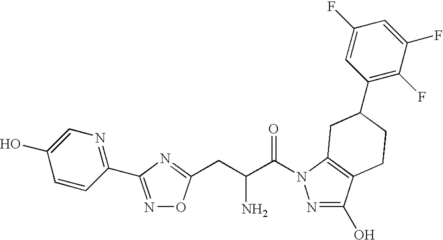

EXAMPLE 14 was prepared in a similar manner to EXAMPLE 13 (Scheme 5) utilizing the intermediate prepared for EXAMPLE 11. ¹H NMR (DMSO-d₆, 500 MHz) δ 10.76 (br s, 1H), 8.65 (br s, 1H), 8.26 (dd, 1H), 7.90 (d, 1H), 7.87 (t, 1H), 7.45-7.40 (m, 1H), 7.38-7.32 (m, 1H), 7.20-7.15 (m, 2H), 5.29 (q, 1H), 3.79-3.55 (3H), 3.33-3.25 (m, 2H), 2.92-2.90 (m, 1H), 2.76-2.66 (m, 2H), 2.46-2.39 (m, 2H), 1.96-1.91 (m, 2H); LCMS m/z 501 (M+1).

### Example 15

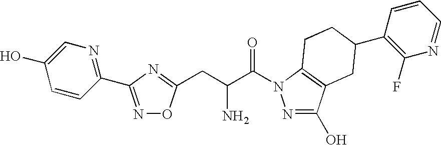

EXAMPLE 15 was prepared in a similar manner to EXAMPLE 13 (Scheme 5) utilizing the intermediate prepared for EXAMPLE 10. ¹H NMR (DMSO-d₆, 500 MHz) δ 11.43 (s, 1H), 10.71 (s, 1H), 8.62 (s, 2H), 8.27 (d, 1H), 8.13 (d, 1H), 7.93-7.87 (m, 2H), 7.36-7.33 (m, 2H), 5.30 (s, 1H), 3.78-3.64 (, 2H), 3.15-3.11 (m, 2H), 2.98-2.93 (m, 1H), 2.62-2.53 (m, 2H), 2.09-2.08 (m, 2H), 1.98-1.94 (m, 1H); LCMS m/z 466 (M+1).

### Example 16

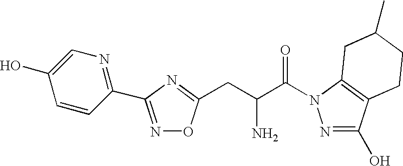

EXAMPLE 16 as prepared in a similar manner to EXAMPLE 13 (Scheme 5) utilizing the intermediate prepared for EXAMPLE 2. (major diastereomer) ¹H NMR (DMSO-d₆, 600 MHz δ 11.08 (s, 1H), 8.46 (s, 1H), 7.40 (d, 1H), 6.94 (d, 1H), 5.59-5.56 (m, 1H), 3.61-3.31 (m, 3H), 3.09-3.00 (m, 1H), 2.61-2.56 (m, 1H), 2.36-3.26 (m, 1H), 2.21-2.16 (m, 1H), 2.08-2.06 (m, 1H), 1.88-1.74 (m, 2H), 1.32 (d, 2H) 1.27-1.21 (m, 1H); LCMS m/z 385 (M+1).

### Example 17

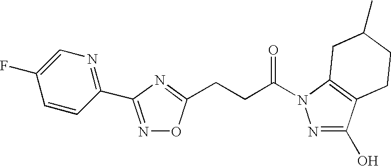

As shown in Scheme 6 a suspension of 5-amino-2-cyano pyridine (20.0 g, 0.168 mol) in HF-pyridine (100 g) in an Erlenmeyer flask cooled to 0° C. was added sodium nitrite (17.4 g, 0.25 mol) in four portions. After 45 min at 0° C. the reaction mixture was stirred at room temperature for 30 min and then heated to 80° C. for 90 min. The reaction mixture was quenched by pouring into ice/water mixture. The resulting mixture was extracted with DCM. The organic layer was dried over anhydrous sodium sulfate, filtered and concentrated to give the fluoropyridine as an orange solid.

To a suspension of this fluoropyridine nitrile intermediate (16.0 g, 0.13 mol) in methanol (200 mL) was added hydroxylamine (9.63 mL, 0.16 mmol, 50% by wt). After stirring the reaction mixture at room temperature for 48 h, it was filtered through a flitted funnel. The precipitate was washed with ether and dried under vacuum to give the N-hydroxy amidine as a yellow solid.

To a suspension of this amidine intermediate (5.32 g, 34.3 mmol) in anhydrous pyridine (10 mL) was added 4-chloro-4-oxo-methyl butyrate (5 mL, 41.2 mmol). The resulting reaction mixture was heated at 120° C. for 2 h. The mixture was cooled to RT and concentrated. The residue was dissolved in ethyl acetate and washed with 1N HCl, water and brine. The organic layer was dried over anhydrous sodium sulfate, filtered and concentrated to give a dark brown solid. This material was purified by Biotage using 25%-60% ethyl acetate-hexanes gradient to give the heterobiaryl intermediate as a light yellow solid.

To a solution of this ester intermediate (900 mg, 3.58 mmol) in THF (4 mL) was added methanol (2 mL) followed by 5N NaOH (1 mL). After 30 min, the reaction mixture was neutralized by the addition of 1N HCl (5 mL). The reaction mixture was concentrated. The residue was extracted with ethyl acetate, and the organic layer was washed with brine, dried over anhydrous sodium sulfate, filtered and concentrated to give a light yellow solid of the carboxylic acid.

EXAMPLE 17 was prepared by reaction of the carboxylic acid described above and the intermediate prepared for EXAMPLE 2. ¹H NMR (DMSO-d₆, 500 MHz) δ 10.95 (br s, 1H), 8.75 (d, 1H), 8.12 (dd, 1H), 7.95-7.91 (m, 1′-1) 0.50 (t, 2H), 3.31 (t, 2H), 3.03 (d, 1H), 2.32-2.21 (m, 3H), 1.75 (br s, 2H), 1.26-1.24 (m, 1H)m 0.99 (d, 3H); LCMS m/z 372 (M+1).

### Example 18

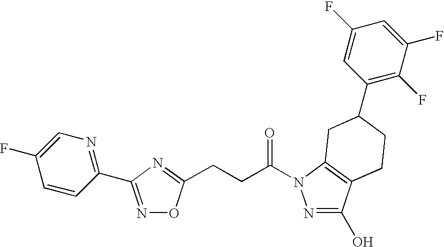

EXAMPLE 18 was prepared in a similar manner to EXAMPLE 17 (Scheme 6) utilizing the intermediate prepared for EXAMPLE 11. ¹H NMR (DMSO-d₆, 500 MHz) δ 11.09 (s, 1H), 8.75 (d, 1H), 8.11 (dd, 1H), 7.93 (dt, 1H), 7.40-7.37 (m, 1H), 7.16-7.13 (m, 1H), 3.59-3.48 (m, 3H), 3.32-3.19 (m, 3H), 2.84 (dd, 1H), 2.42-2.37 (m, 2H), 1.90-1.95 (m, 2H); LCMS m/z 488 (M+1).

### Example 19

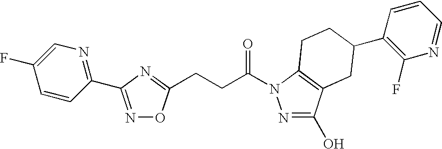

EXAMPLE 19 was prepared in a similar manner to EXAMPLE 17 (Scheme 6) utilizing the intermediate prepared for EXAMPLE 10. NMR (DMSO-d₆, 500 MHz) δ 11.08 (s, 1H), 8.76 (s, 1H), 8.12-8.10 (m, 2H), 7.94-7.91 (m, 2H), 7.33 (br s, 1H), 3.54 (t, 1H), 3.06 (t, 2H), 2.87 (br s, 1H), 2.59-2.38 (m, 4H), 1.98-1.90 (m, 2H); LCMS m/z 453 (M+1).

### Example 20

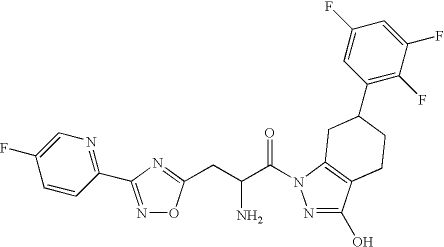

EXAMPLE 20 was prepared in a similar manner to EXAMPLE 13 (Scheme 5) where 5-fluoro-2-hydroxyamidinylpyridine (Scheme 6) was used as an intermediate to obtain the desired product. The intermediate prepared for EXAMPLE 11 was utilized from the synthesis of EXAMPLE 20. (major diastereomer) ¹H NMR (CD₃OD, 500 MHz) δ 8.67 (d, 1H), 8.25 (dd, 1H), 7.87-7.83 (m, 1H), 7.08-7.04 (m, 1H), 6.98-6.96 (m, 1H), 5.50-5.46 (m, 1H), 3.98-3.94 (m, 1H), 3.83 (dd, 1H), 3.47-3.37 (m, 2H), 2.98-2.92 (m, 1H), 2.55-2.42 (m, 2H), 2.06 (d, 1H), 1.95 (d, 1H); LCMS m/z 503 (M+1).

### Example 21

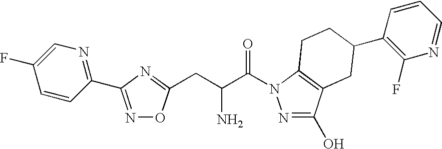

EXAMPLE 21 was prepared in a similar manner to EXAMPLE 20 utilizing the intermediate prepared for EXAMPLE 10. (major diastereomer) ¹H NMR (CD₃OD, 500 MHz) δ 8.69 (s, 1H), 8.28-8.26 (m, 1H), 8.08 (d, 1H), 7.91-7.85 (m, 2H), 7.33-7.30 (m, 1H), 5.49 (q, 1H), 3.96 (dd, 1H), 3.85-3.78 (m, 1H), 3.21-3.18 (m, 2H), 3.04-3.00 (m, 1H), 2.70-2.62 (m, 1H), 2.47-2.44 (m, 1H), 2.17-2.01 (m, 2H); LCMS m/z 468 (M+1).

### Example 22

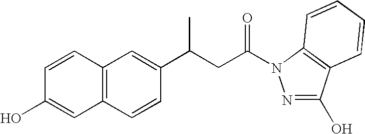

As shown in Scheme 7, commercially available 2-bromo-6-methoxynaphthalene (2.9 g, 12.2 mmol) in anhydrous tetrahydrofuran (20 mL) was chilled to −78° C. under nitrogen, and treated dropwise with a solution of n-butyllithium (1.6 M, 7.6 mL, 12.2 mmol). The reaction mixture was aged for 10 min, and then treated with a solution of 2-butenoic acid (500 mg, 5.8 mmol) in 30 mL of anhydrous tetrahydrofuran under nitrogen atmosphere. The reaction mixture was aged for 1 h at −78° C., quenched with water, partitioned with ethyl acetate, the aqueous phase acidified with 2N HCl to pH 2, washed with ethyl acetate, the organic phase was separated and dried over anhydrous sodium sulfate, and then evaporated under reduced pressure to provide the desired crude carboxylic acid product.

This carboxylic acid intermediate was coupled to commercially available 3-indazolinone in a manner similar to EXAMPLE 1 yielding the desired indazolinone amide intermediate.

This indazolinone amide intermediate (50 mg, 0.14 mmol) was dissolved in anhydrous methylene chloride (3 mL) was chilled to −78° C. under nitrogen, and treated with a solution of boron tribromide (1M, 0.1.4 mL, 0.7 mmol). The reaction mixture was warmed to room temperature, aged for 3 h, and then partitioned between methylene chloride and water, the organic phase was separated and dried over anhydrous sodium sulfate, and then evaporated under reduced pressure. The product was purified via reverse phase HPLC to give EXAMPLE 22. ¹H NMR (CD₃OD, 500 MHz) δ 8.18 (1H, d), 7.62 (1H, d), 7.55-7.44 (4H, m), 7.26 (2H, m), 6.94 (2H, m), 3.54 (1H, m), 3.37 (1H, m), 3.26 (1H, m), 1.35 (3H, d); LCMS m/z 347 (M+1).

### Example 23

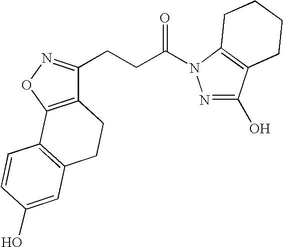

As shown in Scheme 8 a solution of diisopropylamine (5.3 g, 52 mmol) in 200 mL of THF was treated with n-butyllithium (22.4 mL, 56 mmol, 2.5 M in hexane) at −78° C. The resulting solution was stirred at −78° C. for 30 min, and then at RT for an additional 30 min. The solution was re-cooled to −78° C., and to this solution, was added dropwise a solution of tetralone (7.03 g, 39.9 mmol) in 80 mL of THF. After 1 h at −78° C., to the above solution was added methyl 4-chloro-4-oxobutyrate (8.43 g, 6.84 mL, 56 mmol) in one portion. The resulting solution was warmed to 23° C. over 2 h. The solvent was then evaporated, and the residue was diluted with 200 mL of THF/MeOH/water (v:v:v=3:1:1). To this mixture was added 100 mL of lithium hydroxide (1 M in water), and the resulting solution was stirred overnight. After removing some solvent in vacuo, the remaining aqueous layer was extracted with ethyl acetate. The aqueous phase was acidified with HCl until pH=3. The mixture was extracted with ethyl acetate, and the combined organic fractions were dried with sodium sulfate and concentrated in vacuo to give the ketoacid as a grey solid.

To a solution of this ketoacid intermediate (0.72 g, 2.6 mmol) in 15 mL of ethanol were added hydroxylamine hydrochloride (0.22 g, 3.1 mmoL) and triethylamine (320 mg, 0.44 mL, 3.1 mmol). The resulting mixture was heated at reflux for 5 h. After removing ethanol in vacuo, the residue was diluted with ethyl acetate (100 mL) and 1N HCl (20 mL). The aqueous layer was further extracted with 30% of isopropanol in chloroform (2×30 mL). The organic fractions were combined, dried with sodium sulfate and concentrated in vacuo to give the tricycle as a pale yellow solid. This intermediate was dissolved in dichloromethane (20 mL) and boron tribromide (10 mL, 1 M in dichloromethane) was added at 0° C. The resulting dark solution was stirred at room temperature for 4 h before it was quenched with 100 mL of water at 0° C. The mixture was extracted with 30% isopropanol in chloroform. The aqueous layer contained a lot of product as a yellow solid, which was collected by filtration. The aqueous layer was further extracted with 30% isopropanol in chloroform. The organic phase was dried with sodium sulfate and concentrated in vacuo to give the hydroxy product as a yellow solid after reverse phase-HPLC purification.

To a solution of this hydroxy acid intermediate (110 mg, 0.42 mmol) in 15 mL of dichloromethane were added imidazole (87 mg, 1.3 mmol) and tert-butyldimethylsilyl chloride (192 mg, 1.3 mmoL) at RT. The resulting mixture was stirred for 4 h. The mixture was then purified by Biotage to give the desired product as a colorless oil. This carboxylic acid intermediate (7 mg, 0.05 mmol) was dissolved in 2 mL DCM, and oxayl chloride [2M in DCM] (0.14 mmol, 0.7 mL) was added followed by the addition of 2 drops of anhydrous DMF. The reaction was stirred for 30 minutes before being heated to 40° C. and evaporated with a continuous stream of nitrogen. The residue was placed on the vacuum pump for 1 hour then taken up in THF (2 mL) and 5.0 equiv of triethyl amine (0.14 mmol, 0.02 mL) was added. The resulting suspension was stirred for 5 minutes, taken up by syringe and added to a solution of 3-tetrahydro indazolinone (0.13 mmol, 17 mg) dissolved in 2 mL of THF and cooled to 0° C. The reaction mixture was then stirred at 0° C. for 3 hours before quench with saturated ammonium chloride and extraction with ethyl acetate. The combined organic extracts were evaporated under reduced pressure and EXAMPLE 23 was purified by PTLC in 5% MeOH/DCM (Scheme 8). ¹H NMR (CD₃OD, 500 MHz) δ 7.70 9d, 1H), 7.55 (t, 1H), 7.45-7.43 (m, 2H), 7.15 (d, 1H), 7.08-7.04 (m, 1H), 6.68-6.64 (m, 2H), 3.32-3.23 (m, 4H), 2.91-2.86 (m, 4H), 2.81 (br s, 2H), 2.60-2.57 (m, 2H); LCMS ink 380 (M+1).

### Example 24

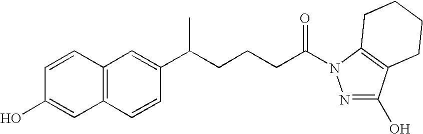

As shown in Scheme 9 the carboxylic acid derivative prepared for EXAMPLE 22 (250 mg, 1.0 mmol) in diethyl ether (15 mL) was added dropwise to a solution of lithium aluminum hydride (76 mg, 2.0 mmol) in 15 mL of anhydrous diethyl ether under nitrogen atmosphere. The reaction mixture was aged, quenched with aqueous Rochelle salt, stirred for an additional 2 h, partitioned between saturated aqueous NaHCO₃ and diethyl ether, the organic phase was separated and dried over anhydrous sodium sulfate, and then evaporated under reduced pressure to provide the crude alcohol product (200 mg). This alcohol (180 mg, 0.75 mmol) was oxidized directly with iodobenzene diacetate (266 mg, 0.83 mmol) and catalytic TEMPO (10%) in methylene chloride solvent (15 mL). The reaction mixture was quenched with aqueous sodium thiosulfate, partitioned with methylene chloride, the organic phase washed with aqueous NaHCO₃, and the organic phase concentrated in vacuo to provide the clean aldehyde product. This crude aldehyde intermediate (180 mg, 0.75 mmol) was combined with methyl (triphenylphosphoranylidene)acetate (376 mg, 1.1 mmol) in toluene (20 mL), and the reaction mixture heated at reflux. The mixture was concentrated in vacuo to a residue which was purified by flash column chromatography (SiO₂, EtOAc/hexanes) to give the desired methyl enoate. This intermediate was dissolved in tetrahydrofuran (20 mL), treated with aqueous 1N NaOH (2 mL), refluxed, the mixture cooled, acidified and extracted with diethyl ether. The organic phase was concentrated in vacuo to provide the clean enoic acid, which was then treated directly with catalytic palladium on carbon in methanol (15 mL), and hydrogenated at 1 atmosphere with a hydrogen-filled balloon. The reaction mixture was filtered over celite and concentrated in vacuo to provide the clean carboxylic acid intermediate.

This intermediate (60 mg, 0.22 mmol) was dissolved in anhydrous methylene chloride (3 mL) was chilled to −78° C. under nitrogen, and treated with a solution of boron tribromide (1M, 1.1 mL, 1.1 mmol). The reaction mixture was warmed to room temperature, aged for 3 h, and then partitioned between methylene chloride and water, the organic phase was separated and dried over anhydrous sodium sulfate, and then evaporated under reduced pressure. The product was purified via reverse phase HPLC to give the desired hydroxyl intermediate.

To a solution of this hydroxy acid intermediate (31 mg, 0.11 mmol) in 5 mL of dichloromethane were added triethylamine (0.1 mL, 0.72 mmol), DMAP (3 mg), and tert-butyldimethylsilyl chloride (54 mg, 0.36 mmoL) at RT. The resulting mixture was stirred for 6 h. The mixture was then purified by PTLC in 10% MeOH/DCM to give the desired silyl-protected product (34 mg). This silyl-protected intermediate (33 mg, 0.09 mmol) was dissolved in 2 mL DCM, and oxalyl chloride [2M in DCM] (0.27 mmol, 0.13 mL) was added followed by the addition of 2 drops of anhydrous DMF. The reaction was stirred for 30 minutes before being heated to 40° C. and evaporated with a continuous stream of nitrogen. The residue was placed on the vacuum pump for 1 hour then taken up in THF (2 mL), cooled to 0° C. and a solution of intermediate A (0.23 mmol, 30 mg) dissolved in 1 mL of THF was added. The reaction mixture was then allowed to slowly warm to room temperature overnight before quench with saturated ammonium chloride and extraction with ethyl acetate. The combined organic'extracts were evaporated under reduced pressure and the product was purified by reverse phase HPLC. This intermediate (7 mg, 0.01 mmol) was then was taken up in methylene chloride (0.5 mL) and trifluoroacetic acid (0.5 mL), and stirred at room temperature for 1 hour before the reaction mixture evaporated under reduced pressure. Purification of the reaction residue by PTLC in 10% MeOH/DCM gave EXAMPLE 24. ¹H NMR (DMSO-d₆, 500 MHz) δ 7.67 (d, 1H), 7.55-7.48 (m, 1H), 7.44 (s, 1H), 7.20 (d, 1H), 7.05 (t, 1H), 6.98-6.91 (m, 1H), 2.97 (t, 2H), 2.80-2.78 (m, 1H), 1.76-1.55 (m, 4H), 1.36) d, 3H); LCMS m/z 379 (M+1).

### Example 25

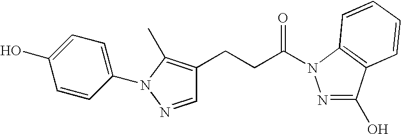

As shown in Scheme 10 trimethylphosphonoacetate (582 mg, 3.19 mmol), in THF (40 mL) at 0° C. was added n-butyl lithium (1.6 M, 3.49 mmol, 2.18 mL). The resulting reaction mixture was stirred at 0° C. for 30 minutes before 1-(4-methoxyphenyl)-5-methyl-1H-pyrazole-4-carboxaldehyde (628 mg, 2.91 mmol) in THF (10 mL) was added and the reaction mixture was allowed to slowly warm to room temperature overnight. The reaction was then diluted with water and extracted with ethyl acetate. Evaporation under reduced pressure gave a residue that purified by column chromatography (SiO₂) yielding the desired α,β-unsaturated ester product.

This α,β-unsaturated ester intermediate (740 mg) was dissolved in THF (5 mL0, MeOH (5 mL), and 1N aqueous LiOH solution (5 mL). After 3 hours the reaction mixture was acidified to pH 4 and extracted with ethyl acetate. Evaporation of the combined organic extracts gave the desired carboxylic acid product as a white solid that was used without further purification.

This intermediate (250 mg, 1.0 mmol) and Pd/C (10%, 50 mg) in 20 mL of methanol was stirred under 1 atm of hydrogen gas (balloon) for 2 hrs. The slurry was filtered and concentrated in vacuo. The desired saturated product was used without further purification.

This intermediate (30 mg, 0.11 mmol) was dissolved in dichloromethane (2.5 mL) and boron tribromide (0.57 mL, 1 M in dichloromethane) was added at 0° C. The resulting dark solution was stirred at room temperature for 4 h before it was quenched with 10 mL of water at 0° C. The mixture was extracted with 30% isopropanol in chloroform. The organic phase was dried with sodium sulfate and concentrated in vacuo to give the desired hydroxylproduct as a white solid after reverse phase-HPLC purification.

To a solution of this hydroxy acid intermediate (28 mg, 0.1 mmol) in 2 mL of dichloromethane were added triethylamine (0.11 mL, 0.74 mmol), DMAP (3 mg), and tert-butyldimethylsilyl chloride (50 mg, 0.33 mmoL) at RT. The resulting mixture was stirred for 6 h before being quenched with water and extracted with ethyl acetate. The desired crude silyl product was used without further purification.

This carboxylic acid intermediate (10 mg, 0.03 mmol) was dissolved in 2 mL DCM, and oxayl chloride [2M in DCM] (0.09 mmol, 0.04 mL) was added followed by the addition of 1 drop of anhydrous DMF. The reaction was stirred for 30 minutes before being heated to 40° C. and evaporated with a continuous stream of nitrogen. The residue was placed on the vacuum pump for 1 hour then taken up in DCM (2 mL). The resulting suspension was cooled to 0° C. and 3-indazolinone (0.08 mmol, 10 mg) was added. The reaction mixture was then allowed to slowly warm to room temperature over 15 hours before quench with saturated aqueous ammonium chloride and extraction with ethyl acetate. The combined organic extracts were evaporated under reduced pressure and EXAMPLE 25 was purified by reverse phase HPLC. NMR (CD₃OD, 500 MHz) δ 8.24 (d, 1H), 7.64 (d, 1H), 7.50 (t, 1H), 7.40 (s, 1H), 7.29 (t, 1H), 7.09 (d, 2H), 6.80 (d, 2H), 3.23 (t, 2H), 2.89 (t, 2H), 2.12 (s, 3H); LCMS m/z 363 (M+1).

### Biological Assays

The activity of the compounds of the present invention regarding niacin receptor affinity and function can be evaluated using the following assays:

### ³H-Niacin Binding Assay:

1. Membrane: Membrane preps are stored in liquid nitrogen in:
20 mM HEPES, pH 7.4
0.1 mM EDTA

Thaw receptor membranes quickly and place on ice. Resuspend by pipetting up and down vigorously, pool all tubes, and mix well. Use clean human at 15 μg/well, clean mouse at 10 ug/well, dirty preps at 30 ug/well.
1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.
   2. Wash buffer and dilution buffer: Make 10 liters of ice-cold Binding Buffer:
20 mM HEPES, pH 7.4
1 mM MgCl₂
0.01% CHAPS (w/v)
use molecular grade or ddH₂O water
   3. [5,6⁻³H]-nicotinic acid: American Radiolabeled Chemicals, Inc. (cat #ART-689). Stock is ˜50 Ci/mmol, 1 mCi/ml, 1 ml total in ethanol→20 μM

Make an intermediate ³H-niacin working solution containing 7.5% EtOH and 0.25 μM tracer. 40 μL of this will be diluted into 200 μl, total in each well→1.5% EtOH, 50 nM tracer final.

4. Unlabeled nicotinic acid:
Make 100 mM, 10 mM, and 80 μM stocks; store at −20° C. Dilute in DMSO.

### 5. Preparing Plates:

1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 μl:40 μl).
3) Add 1954, binding buffer to all wells of Intermediate Plates to create working solutions (250 μM 0). There will be one Intermediate Plate for each Drug Plate.
4) Transfer 54 from Drug Plate to the Intermediate Plate. Mix 4-5 times.

### 6. Procedure:

1) Add 140 μL of appropriate diluted 19CD membrane to every well. There will be three plates for each drug plate: one human, one human+serum, one mouse.
2) Add 20 μL of compound from the appropriate intermediate plate.
3) Add 40 μL of 0.25 μM ³H-nicotinic acid to all wells.
4) Seal plates, cover with aluminum foil, and shake at RT for 3-4 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of 1 nM to about 25 μM.

³⁵S-GTPγS binding assay:

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation

### Materials:

CHO-K1 cell culture medium: F-12 Kaighn's Modified Cell Culture Medium with 10% FBS, 2 mM L-Glutamine, 1 mM Sodium Pyruvate and 400 μg/ml G418

### Membrane Scrape Buffer: 20 mM HEPES

10 mM EDTA, pH 7.4

### Membrane Wash Buffer: 20 mM HEPES

0.1 mM EDTA, pH 7.4
   Protease Inhibitor Cocktail: P-8340, (Sigma, St. Louis, Mo.)

### Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 504 Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay

### Materials:

Guanosine 5′-diphosphate sodium salt (GDP, Sigma-Aldrich Catalog #87127)
Guanosine 5′-[γ³⁵S] thiotriphosphate, triethylammonium salt ([³⁵S]GTPγS, Amersham

### Biosciences Catalog #SJ1320, ˜1000Ci/mmol)

96 well Scintiplates (Perkin-Elmer #1450-501)

### Binding Buffer: 20 mM HEPES, pH 7.4

100 mM NaCl
10 mM MgCl₂
   GDP Buffer: binding buffer plus GDP, ranging from 0.4 to 40 μM, make fresh before assay

### Procedure:

(total assay volume=100 μ̅well)

25 μL GDP buffer with or without compounds (final GDP 10 μM—so use 40 μM stock)

50 μL membrane in binding buffer (0.4 mg protein/mL)

25 μL [³⁵S]GTPγS in binding buffer. This is made by adding 5 μl [³⁵S]GTPγS stock into

10 mL binding buffer (This buffer has no GDP)
Thaw compound plates to be screened (daughter plates with 54 compound @ 2 M in 100% DMSO).
Dilute the 2 mM compounds 1:50 with 2454 GDP buffer to 40 μM in 2% DMSO. (Note: the concentration of GDP in the GDP buffer depends on the receptor and should be optimized to obtain maximal signal to noise; 40 μM).
Thaw frozen membrane pellet on ice. (Note: they are really membranes at this point, the cells were broken in the hypotonic buffer without any salt during the membrane prep step, and most cellular proteins were washed away).
Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
Add 25 μL compounds in GDP buffer per well to Scintiplate.
Add 50 μL of membranes per well to Scintiplate.
Pre-incubate for 5-10 minutes at room temperature. (cover plates with foil since compounds may be light sensitive).
Add 25 μL of diluted [³⁵S]GTPγS. Incubate on shaker (Lab-Line model #1314, shake at setting of 4) for 60 minutes at room temperature. Cover the plates with foil since some compounds might be light sensitive.
Assay is stopped by spinning plates sealed with plate covers at 2500 rpm for 20 minutes at 22° C.
Read on TopCount NXT scintillation counter—35S protocol.

The compounds of the invention generally have an EC₅₀ in the functional in vitro GTP'yS binding assay within the range of about less than 1 μM to as high as about 100 μM.

### Flushing Via Laser Doppler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

### Materials and Methods—Laser Doppler Pirimed PimII; Niacin (Sigma); Nembutal (Abbott Labs).

All patents, patent applications and publications that are cited herein are hereby incorporated by reference in their entirety. While certain preferred embodiments have been described herein in detail, numerous alternative embodiments are seen as falling within the scope of the invention.

1. A compound represented by formula I: 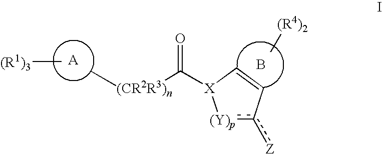
or a pharmaceutically acceptable salt or solvate thereof, wherein:
X represents a nitrogen or carbon atom;
Y represents C or N, such that when Y represents nitrogen, the nitrogen atom may be optionally substituted with H or R⁶ wherein:
R⁶ represents C₁₋₃alkyl optionally substituted with 1-3 halo groups;
and when Y represents a carbon atom, the carbon atom may be substituted with hydrogen or halo;
p represents an integer of from 1 to 2, such that when p represents 2, no more than one Y represents a nitrogen atom;
the dashed lines represent optional bonds;
when the dashed line to Z represents a bond that is present, Z is selected from O, S and NH and the dashed line to (Y)ₚ represents a bond that is absent;
when the dashed line to Z represents a bond that is absent, the dashed line to (Y)ₚ represents a bond that is present and Z represents a group selected from OH, SH, NH₂, CO₂H and SO₃H;
ring B represents phenyl, a 5-7 membered carbocycle, or a 5-6 membered heteroaryl, heterocyclic or partially aromatic heterocyclic group, said heteroaryl, heterocyclic and partially aromatic heterocyclic groups containing at least one heteroatom selected from O, S and N, and optionally containing 1 additional N atom, with up to 2 heteroatoms being present;
each R⁴ is H or halo, or is selected from the group consisting of:
a) a phenyl or a 5-6 membered heteroaryl group containing 1 heteroatom selected from O, S and N, and optionally containing 1-3 additional N atoms, said phenyl and heteroaryl groups being optionally substituted with 1-3 substituents, 1-3 of which are halo, and 0-1 of which are selected from: OH, NH₂, C₁₋₃alkyl, C₁₋₃alkoxy, haloC₁₋₃alkyl and haloC₁₋₃alkoxy; and
(b) C₁₋₃alkyl optionally substituted with 1-3 substituent groups, 1-3 of which are halo atoms, and 0-1 of which are selected from the group consisting of: OH, OC₁₋₃alkyl, NH₂, NHC₁₋₃alkyl, N(C₁₋₃alkyl)₂, CN, NO₂, Hetcy, phenyl and a 5-6 membered heteroaryl group containing 1 heteroatom selected from O, S and N, and optionally containing 1-3 additional N atoms, said phenyl and heteroaryl groups being optionally substituted with 1-3 substituents, 1-3 of which are halo, and 0-1 of which are selected from: OH, NH₂, C₁₋₃alkyl, C₁₋₃alkoxy, haloC₁₋₃alkyl and haloC₁₋₃alkoxy;
ring A represents a 6-10 membered aryl, a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present;
R² and R³ are independently H, C₁₋₃alkyl, haloC₁₋₃alkyl, OC₁₋₃alkyl, haloC₁₋₃alkoxy, OH, NH₂ or F;
n represents an integer of from 1 to 5;
each R¹ is H or is selected from the group consisting of:
a) halo, OH, CO₂H, CN, NH₂, S(O)₀₋₂R^{e} wherein R^{e} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;
c) Hetcy, NHC₁₋₄alkyl and N(C₁₋₄alkyl)₂, the alkyl portions of which are optionally substituted as set forth in (b) above;
d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;
e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
(b) Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;
f) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of:
i) OH; CO₂H; CN; NH₂; S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in (b) above;
iv) NR′C(O)R″, NR′SO₂R″, NR′CO₂R″ and NR′C(O)NR″R′″ wherein R′, R″ and R′″ are as described above.

2. A compound in accordance with claim 1 wherein Y represents a nitrogen atom unsubstituted or substituted with R⁶.

3. A compound in accordance with claim 1 wherein Y represents a carbon atom.

4. A compound in accordance with claim 1 wherein p represents 1.

5. A compound in accordance with claim 1 wherein p represents 2.

6. A compound in accordance with claim 1 wherein the dashed lines represent optional bonds;
when the dashed line to Z represents a bond that is present, Z represents O,
and when the dashed line to Z represents a bond that is absent, the dashed line to (Y)ₚ represents a bond that is present and Z represents OH.

7. A compound in accordance with claim 1 wherein ring B represents a phenyl ring or a 5-7 membered carbocycle.

8. (canceled)

9. (canceled)

10. A compound in accordance with claim 1 wherein ring A represents a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present.

11. A compound in accordance with claim 10 wherein ring A represents a 5-13 membered heteroaryl group, containing at least one heteroatom selected from O, S and N, and optionally containing 1 other heteroatom selected from O and S, and optionally containing 1-3 additional N atoms, with up to 5 heteroatoms being present.

12. (canceled)

13. A compound in accordance with claim 11 wherein ring A represents a 5 membered heteroaryl group selected from the group consisting of: oxadiazole, thiazole, pyrazole, triazole and oxazole.

14. A compound in accordance with claim 13 wherein ring A represents a 5 membered heteroaryl group selected from the group consisting of: oxadiazole and pyrazole.

15. A compound in accordance with claim 1 wherein n represents 2, 3 or 4.

16. A compound in accordance with claim 15 wherein n represents 2.

17. A compound in accordance with claim 1 wherein each R² and R³ are selected from the group consisting of: H, C₁₋₃alkyl, OH and NH₂, with no more than one being OH or NH₂.

18. A compound in accordance with claim 17 wherein R² and R³ are selected from the group consisting of: H, C₁₋₃alkyl and NH₂, with no more than one being NH₂.

19. (canceled)

20. A compound in accordance with claim 1 wherein each R¹ is H or is selected from the group consisting of:
a) halo, OH and NH₂,
b) NR′SO₂R″ wherein R′ represents H, C₁₋₃alkyl or haloC₁₋₃alkyl, and R″ represents Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups; and
c) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of OH and NH₂.

21. A compound in accordance with claim 20 wherein each R¹ is H or is selected from the group consisting of:
a) halo or OH;
b) NR′SO₂R″ wherein R′ represents H, C₁₋₃alkyl or haloC₁₋₃alkyl, and R″ represents Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups; and
c) phenyl or a 5-6 membered heteroaryl group attached at any available point and being optionally substituted with 1-3 halo, methyl or halomethyl groups, or 1 moiety selected from the group consisting of OH and NH₂.

22. A compound in accordance with claim 21 wherein each R¹ is H or is selected from the group consisting of:
a) halo or OH and
b) phenyl or a 5-6 membered heteroaryl group attached at any available point and being optionally substituted with 1-3 halo, methyl or halomethyl groups, or 1 moiety selected from the group consisting of OH and NH₂.

23. A compound in accordance with claim 1 wherein:
Y represents a carbon or nitrogen atom;
p represents 1 or 2, such that when p represents 2, no more than one Y represents a nitrogen atom;
the dashed lines represent optional bonds;
when the dashed line to Z represents a bond that is present, Z represents O;
and the dashed line to (Y)ₚ represents a bond that is absent;
when the dashed line to Z represents a bond that is absent, the dashed line to (Y)ₚ represents a bond that is present and Z represents OH;
ring B represents a phenyl ring or a 5-7 membered carbocycle;
ring A represents a 5-13 membered heteroaryl or a partially aromatic heterocyclic group, said heteroaryl and partially aromatic heterocyclic group containing at least one heteroatom selected from O, S and N, and optionally containing 1 other heteroatom selected from O and S, and optionally containing 1-3 additional N atoms, with up to 5 heteroatoms being present;
n represents 2, 3 or 4;
each R² and R³ are selected from the group consisting of: H, C₁₋₃alkyl, OH and NH₂, with no more than one being OH or NH₂; and
each R¹ is H or is selected from the group consisting of:
a) halo, OH and NH₂,
b) NR′SO₂R″ wherein R′ represents H, C₁₋₃alkyl or haloC₁₋₃alkyl, and R″ represents Hetcy, Aryl or HAR, said Aryl and HAR being further optionally substituted with 1-3 halo, C₁₋₄alkyl, C₁₋₄alkoxy, haloC₁₋₄alkyl and haloC₁₋₄alkoxy groups; and
c) phenyl or a 5-6 membered heteroaryl or heterocyclic group attached at any available point and being optionally substituted with 1-3 halo, C₁₋₃alkyl or haloC₁₋₃alkyl groups, or 1-2 OC₁₋₃alkyl or haloOC₁₋₃alkyl groups, or 1 moiety selected from the group consisting of OH and NH₂.

24. A compound in accordance with claim 1 selected from table 1 below:
TABLE 1
Compound 1 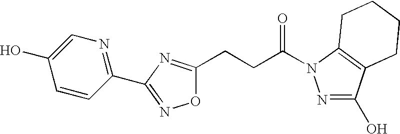
Compound 2 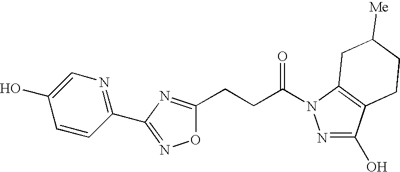
Compound 3 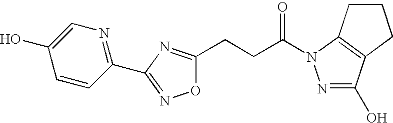
Compound 4 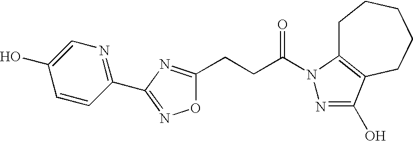
Compound 5 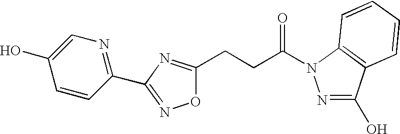
Compound 6 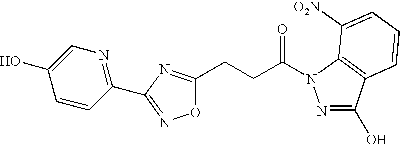
Compound 7 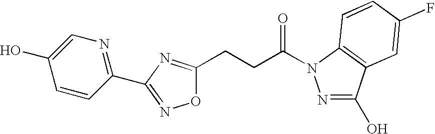
Compound 8 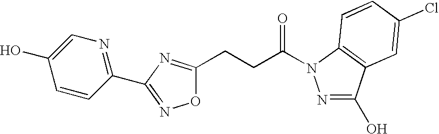
Compound 9 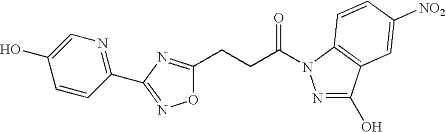
Compound 10 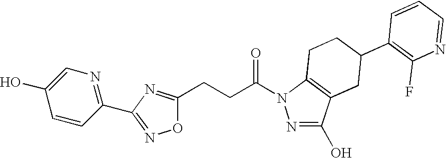
Compound 11 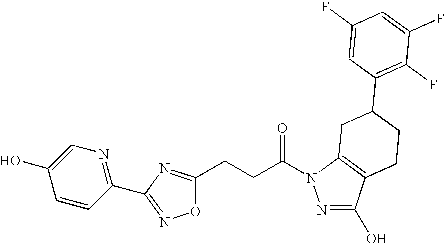
Compound 12 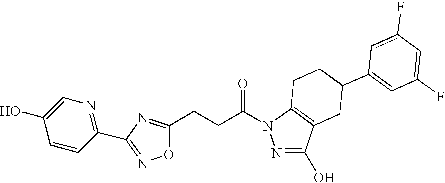
Compound 13 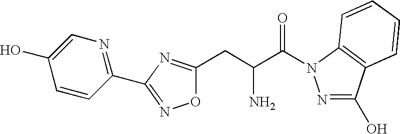
Compound 14 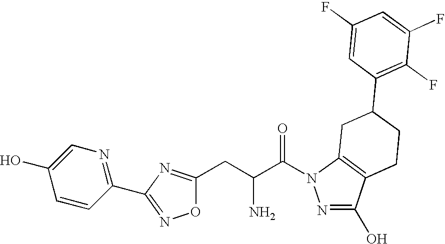
Compound 15 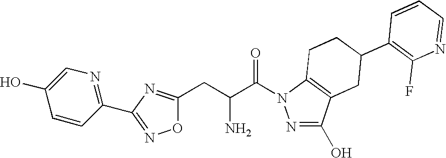
Compound 16 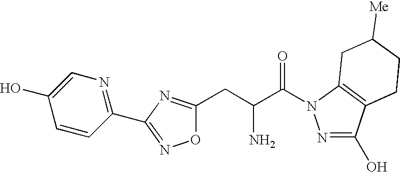
Compound 17 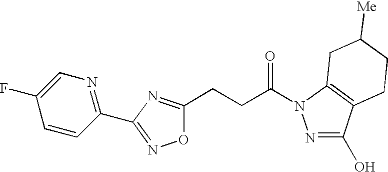
Compound 18 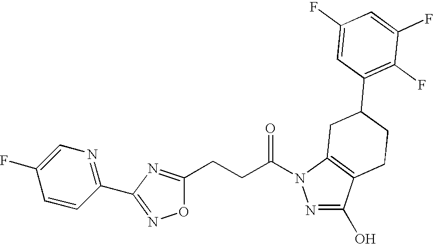
Compound 19 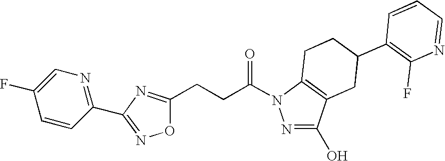
Compound 20 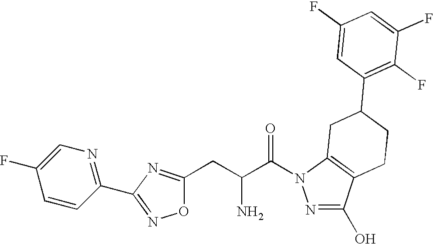
Compound 21 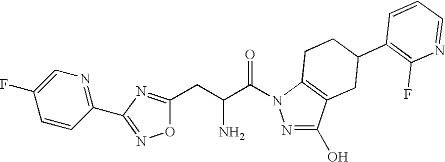
Compound 22 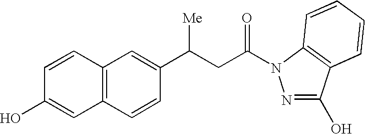
Compound 23 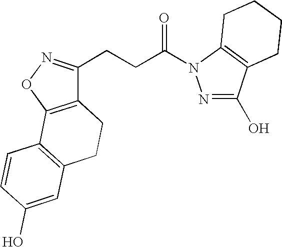
Compound 24 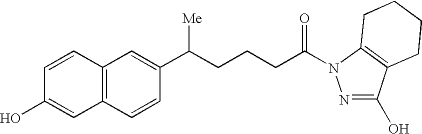
Compound 25 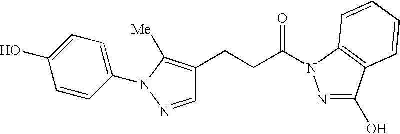
or a pharmaceutically acceptable salt or solvate thereof.

25. A pharmaceutical composition comprising a compound in accordance with claim 1 in combination with a pharmaceutically acceptable carrier.

26. A method of treating atherosclerosis, dyslipidemia, diabetes, metabolic syndrome or a related condition in a human patient in need of such treatment comprising administering to the patient a compound of claim 1 in an amount that is effective for treating atherosclerosis.

27. (canceled)

28. (canceled)

29. (canceled)

30. A method of treating atherosclerosis, dyslipidemias, diabetes, metabolic syndrome or a related condition in a human patient in need of such treatment, comprising administering to the patient a compound of claim 1 and a DP receptor antagonist, said compounds being administered in an amount that is effective to treat atherosclerosis, dyslipidemia, diabetes or a related condition in the absence of substantial flushing.
